(19) United States
(12) Patent Application Publication
(10) Pub. No.: US 2022/0389375 A1
(43) Pub. Date: Dec. 8, 2022

(54) Extracellular Matrix Gels, and Organoid Cultures Comprising the Same

(71) Applicant: UCL BUSINESS LTD., London (GB)

(72) Inventors: Paolo DE COPPI, London (GB); Nicola ELVASSORE, London (GB); Giovanni Giuseppe GIOBBE, London (GB); Claire CROWLEY, London (GB)

(73) Assignee: UCL BUSINESS LTD., London (GB)

(21) Appl. No.: 17/775,172

(22) PCT Filed: Nov. 2, 2020
(86) PCT No.: PCT/EP2020/080659

§ 371 Date: May 6, 2022

(30) Foreign Application Priority Data
Nov. 6, 2019 (GB) 1916135.5

(51) Int. Cl. C12N 5/00; A61K 35/39; C12N 5/071; A61K 35/38

(52) U.S. Cl. CPC C12N 5/0068; A61K 35/39; C12N 5/0677; C12N 5/0679; A61K 35/38; C12N 2533/90; C12N 2533/30

(57) ABSTRACT

The invention concerns novel methods and materials for preparing extracellular matrix (ECM) powder pre-gel and gel solutions, for example for use in organoid culture. The ECM gels demonstrate excellent physiological and mechanical properties while having the proteomic signature of endoderm tissue with specific enrichment of key ECM proteins relevant to organoid formation.

### TECHNICAL FIELD

This invention relates to Extracellular matrix (ECM) gels (or “hydrogels”, as used interchangeably herein) and pre-gels. Such hydrogels find use in culturing human- and animal-derived organoids, and in methods of delivering such organoid cultures.

### BACKGROUND ART

Organoids are three-dimensional multicellular constructs which are able to maintain their stemness throughout unlimited expansion, and functionally differentiate to mature phenotypes¹. As a consequence, organoids are a promising cell source for tissue regeneration, tissue repair, and could be applied as a therapeutic tool for various disease models.

Mouse organoids have been derived from various tissue types and extensively investigated both in vitro^{2,3} and in vivo⁴. Human organoids from different endodermal tissues have been extensively investigated in vitro^{5,6}. Fewer studies have reported the investigation of human organoids for in vivo applications. Still, these models hold great promise for regenerative medicine^{7,8}.

Organoids are commonly cultured in 3D hydrogel systems, which are highly hydrated polymer networks. However, to apply these culture systems in a clinical environment, various limitations must be overcome.

One essential constraint relates to the ability to expand these organoids in conditions that are GMP(good manufacturing practice)-compliant. Most organoids are generated by the simple expansion of stem cells in 3D structures of extracellular matrix-derived proteins. An example of such a structure is Matrigel, which is the trade name for a gelatinous protein mixture secreted by Engelbreth-Holm-Swarm (EHS) mouse sarcoma cells. Matrigel resembles elements of the complex extracellular environment found in many tissues and is used by cell biologists as a substrate for culturing cells in vitro.

However, such a substrate may comprise poorly defined environmental signalling. In addition, due to its derivation from mouse sarcoma, Matrigel will provoke an immune response if used in other species, making it not suitable for human in vivo translation.

Attempts to produce artificial matrices that could overcome these limitations for clinical translation have so far been demonstrated in mouse⁹ and, recently, in human organoids¹⁰. However, it is a real challenge to define all the relevant information necessary to instruct specific tissue remodeling and regeneration. This is also related to the partial information in the literature about the biochemical signature of tissue-specific ECM^{11,12}. As a consequence, synthetic matrices can only partially reproduce some of the native ECM features which commonly include adhesion signals and proteo-cleavable structures.

It can thus be seen that there remains a need for 3D structures capable of culturing organoids, which can reproduce all or many of the native ECM features, while also being potentially GMP-compliant and capable of being translated into a clinical environment.

### DISCLOSURE OF THE INVENTION

The use of naturally derived materials from decellularized tissues (DT) represent excellent candidates for clinical translation. Indeed, DT have been already used clinically, for example as cardiac valve substitutions and for the patch repair of large surgical defects^{13,14}.

Additionally, cell-laden DT efficiently promote in vivo tissue regeneration as demonstrated both by pre-clinical data and experimental human transplantation. These processes suggest that ECM, which can be derived from DT, not only provides a structural support, but also delivers biochemical signals that are fundamental to assisting the regeneration process¹⁵. Hydrogels derived from decellularized tissues potentially have the advantage of providing the cells with all the information they need for their growth and expansion, while also being GMP-compliant¹⁶.

It has been previously shown (Onofri, F. (2016); Master's Degree Thesis; “Development of an extracellular matrix hydrogel for intestine tissue engineering”; Universitá Degli Studi Di Padova) that decellularized porcine small intestinal (SI) mucosa/submucosa¹⁷ can be used in certain protocols to obtain an extracellular matrix self-gelling hydrogel. However, said protocols have also been shown to be incapable, in some conditions, of maintaining long-term organoid culture in vitro.

The inventors have now surprisingly shown that using the protocols disclosed herein, ECM gels can be provided that have physiological ranges and mechanical properties comparable to commercially available gels but nevertheless have the proteomic signature of endoderm tissue with specific enrichment of key ECM proteins relevant to organoid formation. They have demonstrated that ECM gels of the invention are capable of directing and influencing cell behaviour in vitro and in vivo. Furthermore, the inventors have demonstrated that the ECM gels of the invention can support the culture not only of intestinal organoids, but also cells derived from other endodermal derived tissue such as liver, stomach and pancreas, both of mouse and human origin

Furthermore, the inventors have shown that angiogenesis occurred already at 2.5 weeks post-transplantation in vivo, which highlights the potential future clinical applications of this ECM gel.

Furthermore, the inventors have now also surprisingly shown that with the protocols disclosed herein, ECM gels which comprise synthetic polymers suitable for monolayer organoid growth, can be provided.

The ECM-derived hydrogels of the invention can be used in the future for organoid transplantation in clinically relevant environments.

It could not have been predicted that the disclosed protocols could result in ECM gels and organoid cultures capable of displaying such advantageous characteristics in vitro and in vivo.

In one aspect the invention provides a method of preparing an extracellular matrix powder pre-gel solution (“ECM pre-gel” or “pre-gel solution”), the method comprising:
(a) providing decellularised tissue;
(b) processing the decellularised tissue to derive ECM powder from said tissue; and
(c) digesting the powder at a concentration of 1 mg/mL to 8 mg/mL in a proteolytic solution, thereby forming said ECM pre-gel.

In a further aspect, the invention provides a method of preparing an extracellular matrix powder gel solution (“ECM gel” or “gel solution”) prepared by the following steps:
(i) forming an ECM pre-gel by a method of the invention, and
(ii) neutralising the ECM pre-gel to form a gel solution with a pH of 6.8 to 7.7, thereby forming said ECM gel.

In a further aspect, the invention provides an ECM gel prepared by the following steps:
(a) providing decellularised tissue;
(b) processing the decellularised tissue to derive ECM powder from said tissue;
(c) digesting the powder at a concentration of 1-20 mg/mL, or optionally 2-20 mg/mL, or optionally 5-15 mg/mL, or optionally 10 mg/mL, in a proteolytic solution, thereby forming an ECM pre-gel;
(d) neutralising the ECM pre-gel to form a gel solution with a pH of 6.8 to 7.7, thereby forming said ECM pre-gel; and
(e) allowing the ECM pre-gel to gelate; and
(f) mixing the ECM pre-gel with synthetic pre-polymers.

In a further aspect, the invention provides an ECM pre-gel and ECM gel, prepared by the appropriate methods of the invention.

In a further aspect, the invention provides an organoid culture, comprising:
organoids, pieces of organoids, or organoid cell pellets; and
an ECM gel of the invention.

In a further aspect, the invention provides an organoid culture comprising:
organoids, pieces of organoids, or organoid cell pellets; and
an extracellular matrix powder gel solution (“ECM gel” or “gel solution”) comprising
ECM powder at a concentration of between 1 mg/mL and 8 mg/mL.

In a further aspect, the invention provides a method of preparing an organoid culture, comprising mixing:
organoids, pieces of organoids, or organoid cell pellets; and an ECM gel of the invention;
thereby forming said culture.

In a further aspect, the invention provides a method of in vivo delivery of an organoid culture to a subject, comprising administering to the subject a culture of the invention, or a culture obtained by a method of the invention.

In a further aspect, the invention provides a method of treatment of disease in a subject, comprising administering to the subject a culture of the invention, or a culture obtained by a method of the invention.

In a further aspect, the invention provides the culture of the invention for use in any of the methods of treatment of the invention.

In a further aspect, the invention provides the culture of the invention for use in the manufacture of a medicament for any of the methods of treatment of the invention.

The following are embodiments of certain aspects of the invention.

In some embodiments, the decellularised tissue is intestinal tissue, optionally small intestinal tissue. In some embodiments, the small intestinal tissue is the mucosal/submucosal layers of the small intestine. In some embodiments, the tissue is colon tissue, pancreas tissue, oesophageal tissue, gastric tissue, lung tissue, liver tissue, muscle tissue, brain tissue, or cardiac tissue.

In some embodiments, the proteolytic solution is a pepsin solution. In other embodiments, the proteolytic solution is an acidic pepsin solution. In some embodiments, the proteolytic solution is hydrochloric acid (“HCl”) solution. In some embodiments, the proteolytic solution is acetic acid (“CH₃COOH”) solution. In some embodiments, the proteolytic solution is 0.01 to 0.1M HCl. In some embodiments, the proteolytic solution is 0.1 to 0.5 M CH₃COOH In some embodiments, the proteolytic solution comprises 1 mg/mL pepsin. In some embodiments, the proteolytic solution comprises 0.5 mg/mL to 4 mg/mL pepsin. In some embodiments, the proteolytic solution comprises 1 mg/mL pepsin and 0.1 M HCl. In some embodiments, the proteolytic solution comprises 1 mg/mL pepsin and 0.5 M CH₃COOH.

In some embodiments, the powder is digested at a concentration of 2 mg/mL to 8 mg/mL, 2 mg/mL to 6 mg/mL, or at a concentration of 2 mg/mL to 4 mg/mL. In some embodiments, the ECM gel comprises ECM powder at a concentration of 2 mg/mL to 8 mg/mL, 2 mg/mL to 6 mg/mL, or at a concentration of 2 mg/mL to 4 mg/mL.

In some embodiments, prior to step (c), the ECM powder is sterilized by gamma radiation; and/or the decellularised tissue is washed in Milli-Q water and then washed with DNase. In some embodiments, prior to step (c) the ECM powder is sterilised by peracetic acid, ultraviolet radiation or autoclaving. In some embodiments, the sterilisation is performed at −4° C., 0° C., 4° C., 8° C., 12° C., 16° C. or 20° C. In some embodiments, the sterilisation is performed at room temperature.

In some embodiments, prior to step (ii) or step (d), the ECM pre-gel is centrifuged, and precipitated undigested pellets of decellularized tissue, that result from the centrifugation step, are discarded.

In some embodiments, prior to step (ii) or step (d), the ECM pre-gel is freshly prepared at 4° C. for immediate use, or stored at −20° C. In some embodiments, prior to step (ii) or step (d), the ECM pre-gel is freshly prepared and then kept at 4° C. for up to one, two, three or four weeks.

In some embodiments, in step (ii) or step (d), the ECM pre-gel solution is neutralised in Dulbecco's Modified Eagle Medium (DMEM) or DMEM F12, or other culture medium which it is intended to use for subsequent culture steps. The medium may be a 10X medium. In some embodiments, in step (ii) or step (d), the ECM pre-gel solution is neutralised in 10% 10X PBS. In some embodiments, in step (ii) or step (d), the EC pre-gel is neutralised to form a gel solution with a pH of 6.8 to 7.7, 6.9 to 7.6, 7.0 to 7.6, 7.1 to 7.6, 7.2 to 7.6, 7.3 to 7.6, 7.4 to 7.6, 7.5 to 7.6, or 7.5.

In some embodiments, in step (ii) or step (d), neutralisation is performed using cut-end pipette tips.

In some embodiments, in step (e) or step (ii), the ECM pre-gel is allowed for gelate for over 1, 2, 3, 4, 5, 10, 20, 30, 40, 50 or 60 minutes.

In some embodiments, the methods to form an organoid culture further comprise aliquoting droplets of the mixture onto a suitable medium, optionally where the suitable medium is a petri dish, and/or optionally wherein the droplets have a volume of 30-40 μL.

In some embodiments, the organoids or organoid cell pellets are endoderm-derived organoids or organoid cell pellets. In some embodiments, the organoids or organoid cell pellets are human or mouse organoids or organoid cell pellets.

In some embodiments, the endoderm-derived organoids or organoid cell pellets are selected from:
organoids or organoid cell pellets of gastric origin; stomach enteroids or enteroid cell pellets; pediatric stomach enteroids or enteroid cell pellets; ductal (cholangiocyte) organoids or organoid cell pellets; fetal hepatic (hepatocyte) organoids or organoid cell pellets; intestinal stem cells or stem cell pellets; Lgr5+ intestinal stem cells or stem cell pellets; organoid or organoid cell pellets of fetal origin; small intestinal enteroids or enteroid cell pellets; adult cholangiocyte ducts; fetal hepatocyte organoids or organoid cell pellets; human ductal organoids or organoid cell pellets; ductal liver organoids or organoid cell pellets; intestinal organoids or organoid cell pellets; or fetal pancreatic organoids or cell pellets.

In some embodiments, the endoderm-derived organoids or organoid cell pellets are selected from: human adult stomach enteroids or enteroid cell pellets; human adult ductal (liver) organoids or organoid cell pellets; human fetal hepatocyte organoids or organoid cell pellets; human adult small intestinal enteroids or enteroid cell pellets; human fetal small intestinal enteroids or enteroid cell pellets; human fetal pancreatic organoids or cell pellets; mouse adult small intestinal enteroids or enteroid cell pellets.

In some embodiments, the organoid culture can be maintained for more than two passages. In some embodiments, the organoid culture can maintain expression of essential markers after one, two, three, four, five, six, or seven days from culture formation. In some embodiments, the organoid culture can be maintained for more than one, two, three, four, five, six, seven or eight weeks from culture formation.

In some embodiments, the culture does not comprise Matrigel and/or basement membrane extract.

In some embodiments, gene expression in the organoids or organoid cell pellets of the culture is comparable to gene expression in an organoid or organoid cell pellet culture comprising Matrigel.

In some embodiments, the gene is selected from the group consisting of: LGR5, OLFM4, SMOC2, LYZ, BMI1, LRIG1, FABP1, MUC1, MUC3A, MUC5B, EZR, VIL1, MUC12, MUC13, MUC17, MUC20, CHGA, TINAGL1, LTBP4, CRELD1, ECM1, LGALS1, LGALS3, LMAN1, P4HA1, KRT7, KRT8, KRT18, KRT19, EPCAM, SOX9, TACSTD2 (TROP2), ALB, ASGR1, ASGR2, SERPINA1, FABP1, APOA2, ALPI and MUC2.

In some embodiments, gene expression of SOX9 or TROP2 in the organoids or organoid cell pellets of the culture is overexpressed compared to gene expression in an organoid or organoid cell pellet culture comprising Matrigel.

In some embodiments, the culture is conducted for 2-4 days prior to administration.

In some embodiments, the subject is a human subject, a mouse subject, or a mouse model of disease.

In some embodiments, organoid organisation is preserved after 2 weeks, after 3 weeks, after 4 weeks, after 5 weeks, after 6 weeks, after 7 weeks, or after 8 weeks from administration. In some embodiments, the culture comprises matured organoids after 2 weeks, after 3 weeks, after 4 weeks, after 5 weeks, after 6 weeks, after 7 weeks, or after 8 weeks from administration.

In some embodiments, the synthetic pre-polymer comprises poly-acrylamide. In some embodiments, the synthetic pre-polymer comprises polyglycolic acid (PGA), or polylactic acid (PLA).

Some of these aspects and embodiments of the invention will now be described in more detail.

Unless otherwise noted, technical terms are used according to conventional usage. Definitions of common terms in molecular biology may be found in Benjamin Lewin, Genes V, published by Oxford University Press, 1994 (ISBN 0-19-854287-9), Kendrew et al. (eds.), The Encyclopedia of Molecular Biology, published by Blackwell Science Ltd., 1994 (ISBN 0-632-02182-9); and Robert A Meyers (ed.), Molecular Biology and Biotechnology: a Comprehensive Desk Reference, published by VCH Publishers, Inc, 1995 (ISBN 1-56081-569-8)

Terms Used in the Disclosure

Centrifugation

The process whereby a centrifugal force is applied to a mixture, whereby more-dense components of the mixture migrate away from the axis of the centrifuge relative to other less-dense components in the mixture. The force that is applied to the mixture is a function of the speed of the centrifuge rotor, and the radius of the spin. In most applications, the force of the spin will result in a precipitate (a pellet) to gather at the bottom of the centrifuge tube, where the remaining solution is properly called a “supernate” or “supernatant.” In other similar applications, a density-based separation or “gradient centrifugation” technique is used to isolate a particular species from a mixture that contains components that are both more dense and less dense than the desired component.

During the circular motion of a centrifuge rotor, the force that is applied is the product of the radius and the angular velocity of the spin, where the force is traditionally expressed as an acceleration relative to “g,” the standard acceleration due to gravity at the Earth's surface. The centrifugal force that is applied is termed the “relative centrifugal force” (RCF), and is expressed in multiples of “g.”

Conduction

Conduction, in the context of the invention, is the maintenance of an organoid culture in a culturing medium, which may include replacing the culturing medium periodically.

Extracellular Matrix (ECM)

The Extracellular matrix contains secreted products of the resident cells of each tissue and organ. The ECM was initially considered an inert scaffold whose main role is to provide mechanical strength to the tissue, however, today it is accepted as a three-dimensional structure that facilitates the survival of cells by playing an active role in regulating biologic processes, providing physical protection and signals, directing and facilitating cell behaviour such as proliferation, orientation, gene expression, migration and differentiation. It is important that the functional components of the ECM contribute to provide an ideal microenvironment specific to each tissue and organ. For this reason, ECM composition can differ significantly between tissues types. For example the solid calcified structure of the bone differs greatly from the soft and transparent matrix of the cornea.

Fundamentally, the ECM is composed of water, polysaccharides and proteins but each tissue has a specific composition and topology that is the result of the dynamic interactions that occur during tissue development between different cellular components such as fibroblasts, epithelial cells, adipocyte, and the protein environment.

The ECM is responsible for the biochemical and mechanical properties of each organ, including its tensile and compressive strength and elasticity. Moreover it has a buffering action that maintains extracellular homeostasis and water retention. The ECM binds also grow factors (GFs) that interact with cell-surface receptors regulating gene transcriptions and eliciting signal transduction.

Two main classes of macromolecules are present: proteoglycans (PGs) and fibrous proteins PGs make up the majority of the interstitial space in the ECM and appear has a hydrated gel.

Collagen, elastin fibronectin and laminin represent the main fibrous components of the ECM, whose main role is to resist tensile stress. Collagen makes up 30% of the total protein mass of a multicellular animal and so it represents the most abundant component in the ECM. It is responsible for the tensile strength, regulation of cell adhesion and direction of tissue development.

Proteoglycans are formed of a protein core and glycosaminoglycans (GAGs) chains fixed to it. GAGs are negatively charged polysaccharides formed of disaccharide units that might be nonsulphated, like hyaluronic acid which does not form proteoglycan, or sulphated like chondroitin, dermatan, keratan, and heparan sulfates. GAGs due to their polar nature attract cations that in turn will attract water by osmosis. Thus GAGs form a highly hydrated network that gives this gel-like structure to the ECM and allows it to withstand compressive stresses. They also have the role of growth factor reservoir, in fact grow factors, which are molecules that modulate cellular activity, are stabilized and protected from proteolytic degradation by their interaction with GAGs, which modulate a sustained released of these.

Until now it has been difficult to replicate the complexity of ECM in its entirety using biomaterials or mimicking its composition using purified ECM component. Also considering that synthetic biomaterials could potentially mimic the structural environment they could leads to cytotoxic degradation by-products where implanted with a consequent inflammation area. One alternative to synthetic biomaterials is to obtain the native ECM from the tissue by removing the cell content using different types of treatment, which has already be done for some tissue such as bladder, heart, liver, and lung.

Hydrogel

In the context of the invention, the term “gel” and “hydrogel” can be used interchangeably.

Regardless of the widespread use of materials in medicine, many biomaterials aren't good candidates to interface with biological systems and have not been engineered for optimized performance.

Therefore, there is a rising demand to develop optimized materials to solve such problems in different fields as medicine and biology. The cross-linked form of hydrophilic polymers, known as hydrogels, are a class of biomaterials that have demonstrated great potential for this fields.

Hydrogels are three-dimensional networks made of hydrophilic polymers which are crosslinked via covalent bonds or via physical attraction, either intramolecular or intermolecular. They have the ability of absorb and retain a huge amount of water (often more than 90%) and swell without dissolving. Hydrogel are soft and rubbery in the swollen state which make them look like living tissue, for this reason they are used nowadays for many application in tissue engineering. The principal use is as scaffolds that mimic the ECM environment and for 3D cell cultures or as a tool to encapsulate and deliver cells.

In cell transplantation hydrogels have the beneficial ability of create an immunoisolated environment and at the same time allowing the diffusion of nutrients oxygen and metabolic product. As scaffold hydrogels have the ability of mimic the mechanical characteristic of natural tissue and they can be used directly after their preparation or after the creation of a new tissue, they may provide bulk and mechanical structure where cell can be incorporated or suspended in the 3D structure.

There are many different ways in which hydrogel can be classified. First of all they can be physical networks that are the result either of polymer chain entanglements or physical interaction such as ionic interaction, hydrogen bonds or hydrophobic interactions or chemical cross-linked by covalent bonds. These interactions are controlled by the physical condition and can be desegregated by changing ionic strength, pH, temperature, a stress application, or addition of some solutes that compete with the polymeric ligand for the site of affinity on the protein.

Physical hydrogels are not homogeneous due to different factors that can create inhomogeneities such as clusters of molecular entanglements, or hydrophobically or ionically associated domains. Hydrogels are called ‘chemical’ gels when they are covalently-crosslinked networks and they do present permanent properties. It is also possible to classify hydrogel according to their origin which can be natural or synthetic. Synthetic hydrogels such as polyglycolic acid (PGA), polylactic acid (PLA) have the advantages to offer a high degree of the control of the properties such as crosslinking density, or of the properties such as mechanical strength and biodegradation. These synthetic hydrogels may be suitable, alone or in combination, for forming combination hydrogels of certain aspects of the invention.

On the other hand, natural derived hydrogel are considered to demonstrate more adequate biocompatibility, while synthetic hydrogel may elicit significant inflammatory response that can affect the immune response toward the transplanted cells. Moreover synthetic hydrogels lack the endogenous factors such as bioactive molecules that promote cell behaviour and act mainly as a template to permit cell function such as the adhesion, growth, proliferation, differentiation, and ECM secretion of embedded chondrocytes.

Natural hydrogels are typically formed of proteins and ECM components such as collagen, fibrin, hyaluronic acid, or Matrigel, or they can also originate from other biological sources such as plants or animals, for instance chitosan (from crustacean), alginate (from algae) or silk fibrils. These gels have the advantage of already being biocompatible and bioactive. Natural derived hydrogels have the disadvantages of not being completely known in term of composition and so difficult to be reproducible presenting a large batch to batch variation.

Furthermore in the past their physicochemical properties have been difficult to tune, such as their degradation rate being too fast, or having poor mechanical properties.

They have the ability to promote cellular functions thanks to the presence of endogenous factors which can be advantageous for the viability, proliferation, and development of many cell types. However, such scaffolds are complex and often ill-defined, making it difficult to determine exactly which signals are promoting cellular function.

A pre-gel, as used herein, refers to an ECM powder, derived from processed DT, that has been digested in proteolytic solution, as explained herein.

Matrigel

Matrigel comprises basement membrane, or an extract of basement membrane (BME) is a thin, fibrous, extracellular matrix of tissue that separates the lining of an internal or external body surface from underlying connective tissue in animals. This surface may be epithelium, mesothelium and endothelium. In appropriate conditions, it may form a matrix which may be used for culturing cells.

Organisation

As used herein, “organization” can mean cell phenotype, cell disposition in the organoid, or cystic enteroid shape in the case of fetal pancreatic organoids.

Organoid

Organoids are structures which resemble whole organs generated from stem cells through the development of three dimensional culture systems. Organoids are derived from pluripotent stem cells or isolated organ progenitors that differentiate to shape an organ-like tissue exhibiting multiple cell types that self-organize to form the cellular organization of the organ itself. The therapeutic promise of organoids is that they could potentially model developmental diseases, degenerative conditions, and cancer.

Furthermore, organoids that model disease can be used as an alternative system for drug testing that may not only better recapitulate effects in human patients but could also reduce tests on animal. Finally tissues derived in vitro could be generated from patient cells to provide alternative organ replacement strategies.

Unlike current organ transplant treatments, such autologous tissues would not suffer from issues of immunocompetency and rejection.

It has been recently highlighted that in a three-dimensional cultures Lgr5 (marker of stem cell in multiple adult organs of mice and humans) stem cells can grow into ever-expanding epithelial organoids that retain their original organ identity. Single stem cells derived from the patient intestine can be cultured in order to build epithelial structures that maintain hallmarks of the in vivo epithelium.

An organoid cell pellet can be formed by centrifugation of an organoid suspension.

As used herein, “organoid” may refer to stem cells, which may have been derived directly from biopsies, and which have not yet been passaged. As used herein, “piece of organoid” can refer to small cell aggregates of organoids, or single organoid cells.

Passage

A subculture is a new cell or microbiological culture made by transferring some or all cells from a previous culture to fresh growth medium. This action is called a subculturing or a passage. A passage number is the number of times a cell culture has been subcultured.

Small Intestine Tissue

The small intestine (SI) is the part of the gastrointestinal tract which is positioned between the stomach and the colon. It can be divided in 3 different regions: the duodenum, the jejunum, and the ileum. The intestinal wall is composed of 4 distinct layers, which are the mucosa, the submucosa, the muscularis externa, and the serosa, from the lumen to the outside layer.

The mucosa is composed of a layer of epithelial cells organised in villi and crypts, and some connective tissue underlined by a thin layer of smooth muscle. The nutrients absorption is due to the presence of a lot of capillaries passing through the epithelial cell layer, and the immune role is provided by the presence of lymphatic nodules.

The submucosa is mainly composed of connective tissue. The muscularis externa is composed of two different smooth muscular layer, a circular one and a longitudinal one.

Loss in intestinal segments due to congenital diseases or multiple surgical resections due to inflammation or cancer result in the short bowel syndrome, which cause malabsorption, dehydration, electrolyte abnormalities and failure to thrive. The Current therapeutic options for SBS are limited and include bowel lengthening procedures or total parenteral nutrition (TPN) which is not curative. This therapy carries a high morbidity, uses extensive healthcare resources, and diminishes the patient's quality of life.

A promising treatment is small bowel transplantation, however this solution require a donor of appropriate tissue and long term immunosuppression and carry significant risks as morbidity and mortality. A tissue engineering approach could offer a potentially advantageous solution for creating a functional intestinal absorptive area avoiding the complication of currently therapeutic options, since engineering a new intestine in vitro using autologous cells of the patient would solve the issues that exist with allografts. The smallest mucosal unit which can be transplanted are intestinal organoids. These are multicellular crypt-like structures of 20-40 cells which can be isolated from mucosa crypts and that contain the intestine stem cells that comprise the stem cell niche.

In some aspects, the gels and cultures of the invention may be used in the treatment of disease in a subject. In some aspects the gels and cultures of the invention may be used in the manufacture of a medicament for the treatment of disease in a subject. In some aspects the invention provides methods of treatment of disease in a subject, comprising administration of a therapeutically-active amount of the gels and cultures of the invention to the subject.

In some embodiments, the diseases include Intestinal failure (IF); Short bowel syndrome (SBS); Inflammatory bowel disease (IBD); Crohn's disease; Necrotizing enterocolitis (NEC)

Methods for Decellularising Tissue

Decellularising tissues involves removing any cellular material present in the tissue while also conserving the ECM as much as possible. Protocols in the literature to decellularise tissue are well known (Conconi, M. T. et al. Transpl. Int.18, 727-734 (2005); Totonelli, G. et al. Biomaterials 33, 3401-3410 (2012); Baptista, P. M. et al. Hepatology 53, 604-617 (2011).) and they often include a series of detergents and/or enzymatic treatments.

Decellularization of organs and tissues can broadly be divided into physical, chemical, and enzymatic methods. Physical treatments includes agitation, sonication, pressure, frosting and defrosting; these methods destroys cellular membrane allowing the release of cellular contents and making easier their removal from the ECM. Physical treatments are not sufficient alone for the obtainment of a complete decellularization and so they are combined with chemicals treatments. The combination of different approaches is usually adopted for the maximisation of the decellularization effects. Enzymatic treatment, as for example the use of trypsin, and chemicals treatments, as for example the use of ionic solutions and detergents, destroy the cellular membrane and the bonds responsible for the intra and extra-cellular connections. The most effective decellularization protocols present a combination of physical chemical and enzymatic treatment, for example: (i) lysis of the cellular membrane through a physical approach (agitation, pressure, freezing, and de-freezing) or through ionic solutions; (ii) solubilisation of cytoplasm and nuclear components by to chemical detergents; and (iii) separation of cellular components from the ECM through enzymatic tools; These steps can occur, for example, with mechanical agitation in order to increase process efficacy. After the decellularization all the chemical residue must be removed to avoid any adverse response from the host tissue.

Physical Methods

Physical methods that can be used to facilitate the decellularization of tissues include freezing and de-freezing, application of pressure, mechanical agitation and electrophoresis. A rapid freezing of a tissue involve the formation of ice crystals inside the cells which cause cellular lysis after the membrane breaking. Freezing and de-freezing processes effectively destroy cells of organs and tissues but intracellular contents and membrane residues can stay inside if not removed through subsequent processes.

It has been shown that one cycle of freezing and de-freezing is able to lower the immune adverse response, and repeated cycles tend to minimize adverse immune response without involving a significant loss of membrane protein from the tissue. This process has minimal consequences on ECM structure and mechanical properties and on tissue mechanical properties maintenance. Hydrostatic pressure for a relative low time results can be more efficient than detergents and enzymes in cellular removing.

Electroporation is the application of electrical pulsations of microseconds through the tissue and tend to cause the formation of micro pores in the cellular membrane due to the instability of electrical potential. These micro-pores are responsible for homeostasis and cells death.

Chemical Methods

Since all chemical reagents for the cellular removal alter ECM composition one of the objectives of decellularization is the minimization of these adverse effects. Acids and bases provoke or catalyse the hydrolytic degradation of biomolecules, solubilize the cells cytoplasmic component, remove nucleic acids such as DNA and RNA.

For example acetic acid, paracetic acid, ammonium hydroxide can actually destroy cellular membrane and intracellular molecules, but at the same time they dissociate important molecules such as GAG from tissues rich in Collagen.

Hypertonic saline solutions dissociate DNA from proteins, while hypotonic solutions can cause cellular lysis for osmotic effect with minimal consequences on matrix architecture.

In order to increase osmotic effect it is possible to submit the tissue to different cycles of alternate hypotonic and hypertonic solutions; these solutions also comport a rinse of the cellular residues from the tissue.

Non ionic, ionic and Zwitterionic detergents solubilize cellular membrane and dissociate DNA from proteins, are very efficient on removing cellular residues from the decellularized tissue.

Non ionic detergents destroy the lipid-lipid and lipid-protein interaction, leaving the protein-protein interactions intact. Ionic detergents, instead, are capable of destroying also protein-protein bond.

Suitable ionic detergents for decellularising tissue include Triton X-100, Sodium dodecyl sulphate (SDS), sodium deoxycholate (SDC) and triton X-200.

SDC has positive effects on cellular and residues removal but is more aggressive in the removal of the native architecture of the tissue compared to SDS.

Zwitterionic detergents presents some properties which are in between ionic and non-ionic detergents, but have the tendency to provoke protein denaturation. Tri(n-butyl)phosphate (TBP) is an organic solvent which is commonly adopted for the inactivation of virus presents in the blood. Only recently it has been used as a decellularization agent. It presents good decellularization capacity without bringing damages on mechanical properties of ECM, for examples without disrupting collagen fibres. For this reason it appears as a promising decellularization agent.

Finally chelant agents, such as EDTA and EGTA, are molecules that build molecular ring unit which bind and isolate a central metallic ion. Are used to remove cells from protein substrate.

Enzymatic Methods

Enzymatic methods involve the use of enzyme of digestion or nuclease. Trypsin is one of the most adopted protolithic enzyme in the decellularization protocols. It is responsible for the breakage of the peptide bonds at the carboxyl ends of Arg and Lys, and its active at 37° C. and pH=8. ECM components usually have a limitate resistance to trypsin, so it has to be used with caution.

Compared with detergents it is more destructive toward elastin and collagen, but usually is less destructive toward GAGs content. Its action is slow and a complete decellularization with the use of only trypsin is almost impossible since it will require a too long incubation time.

For this reason trypsin is used together with other agents. It destroy the tissue ultrastructure and facilitate the permeation of the others decellularization agents. Nuclease catalyse hydrolysis of DNA and RNA bonds causing their degradation. Also in this case it is important the complete removal of the enzyme after the decellularization process in order to avoid adverse immune response in the host tissue. In the present invention pepsin may be used as an alternative to trypsin.

Decellularization Protocols

The most effective agents for decellularization of each tissue and organ depend upon a number of different factors, including the tissue's cellularity (e.g. liver vs. tendon), density (e.g. dermis vs. adipose tissue), lipid content (e.g. brain vs. urinary bladder), and thickness (e.g. dermis vs. pericardium). Every method and agent for cell removal will alter ECM composition and cause a different degree of ultrastructure disruption which is why it is very important to characterise tissue post-decellularisation when using a new protocol. Different protocols exist and the two most-commonly used are DET (detergent enzymatic treatment) and SDS (sodium dodecyl sulphate).

Both protocols have been shown to be very efficient in removing nuclear remnants and cytoplasmic proteins from dense tissues but tends to disrupt native ultrastructure, removes GAG and growth factors and damages collagen. Before the use of each protocols tissue is frozen at −80° C. for the disruption of cell membrane by intracellular ice crystal. The DET protocol uses SDC (sodium deoxycholate from SIGMA® life science) and can begin with a washing step in MilliQ water (highly purified deionized water) at 4° C. overnight, hypotonic solutions can readily cause cell lysis by simple osmotic effects with minimal changes in matrix molecules and architecture.

The next step can be 4 hour in a 4% weight solution of SDC for the solubilization of cell membranes and dissociation of DNA from proteins. A 30 minutes washing step in PBS (phosphate buffer saline from SIGMA® life science) or Milli-Q water can follow, and then a 3 hours wash in a 1M NaCl, 22.5 mg DNase (Desoxyribonuclease I from bovine pancreas from SIGMA® life science) solution for the elimination of the remaining DNA content. DNase concentration can vary from 500 to 2000 kU depending on the tissue. Enzymes can provide high specificity for removal of cell residues or undesirable ECM constituents. However, complete cell removal by enzymatic treatment alone is difficult and enzyme residues may impair recellularization or evoke an adverse immune response. Nucleases (e.g. DNAses and RNAses) cleave nucleic acid sequences and can therefore aid in removal of nucleotides after cell lysis in tissues.

The removal of ECM proteins and DNA by detergents depend on the amount of time the tissue is exposed to the detergents and enzymes. The speed of decellularisation will also depend on organ subunits, tissue type, and donor age. After the decellularization the tissues can be washed in MilliQ water at 4° C. for three days in order to remove detergents and enzymes which are toxic to the cells.

All the washes can be performed by immersion while being subjected to agitation through a magnetic stirrer, that can lyse cells, but more commonly is used to facilitate chemical exposure and removal of cellular material. The tissue can be cut into small pieces of 1 cm2 to provide as much surface area for the decellularization as possible and avoid the formation of a knot.

Another method involves immersing the tissue in SDS (sodium dodecyl sulphate) for 24 hours. The addition of a detergent such as SDS to a decellularization protocol can make the difference between complete and incomplete cell nuclei removal but has the associated drawback of ultrastructure disruption and growth factor elimination. The protocol involves washing the tissue in MilliQ water (highly purified deionized water) at 4° C. overnight. This causes cell lysis by simple osmotic effects with minimal changes in matrix molecules and architecture. The tissue is then placed in a 0.25% weight solution of SDS followed by a three day washing in MilliQ. This procedure further involved using an immersion method at room temperature with continued agitation. Samples after both one and two days in SDS were investigated.

Further methods for decellularising tissues include those disclosed in the examples herein.

Methods for ECM Derived Hydrogel Methodology

After the decellularization the tissue obtained has to be processed to derive the desired gel. First step consist in the lyophilisation of the tissue by using a freeze-dryer which works by freezing the starting material and then reducing the pressure to allow the sublimation of the frozen water present in the sample from the solid phase to the gas phase. After two days of freeze-drying the samples are milled into a thin powder using a mini-mill (Thomas Wiley® model) with intermediate sized filter (mesh 40).

The powder can be digested with an HCl-pepsin (Pepsin from porcine gastric mucosa from SIGMA® life science) solution. The solution is obtained by adding 1 mg of pepsin powder to each ml of HCl 0.1 M. 1-8 mg of ECM powder can be digested in 1 ml of solution in order to obtain the digestion of the macromolecules that are present and transform them into a more soluble mixture of proteins. The volumes which are usually adopted are 10 ml or 5 ml.

The samples can then be positioned in a shaker at room temperature for 72 hours. After the digestion, the samples can be neutralized using NaOH (sodium hydroxide). A 10% volume of 10×PBS solution can be used as buffer to facilitate the neutralization and to avoid overshooting.

Drops of 5 M and 1 M NaOH can then be used for the neutralization and the pH changing were recorded with a pH-meter. All process can be carried on ice to slow to gelation kinetic so to have a less viscous solution. In fact, the diffusion of NaOH is easily reached in a less viscous solution since to avoid the formation of to many bubbles it was not possible to use a vortex.

When a pH of 7.0 to 7.6 is reached gelation can then be achieved by placing the neutralized solution in an incubator at 37° C. (physiological temperature). Gel can be derived from both (sub)mucosa and whole intestine tissue for DET protocol and from whole intestine for SDS.

Further methods for deriving ECM gel include those disclosed in the examples herein.

Methods for Organoid Culture Formation

Methods for organoid culture formation include those disclosed in the examples herein.

Methods for In Vivo Delivery of an Organoid Culture to a Subject

Methods for in vivo delivery of an organoid culture to a subject include those disclosed in the examples herein.

### FIGURES

FIG. 1—Extracellular matrix hydrogel characterization. (A) The gelation preparation protocol consists of decellularization of the SI mucosa/submucosa, freeze-drying process, milling into a fine powder, gamma-irradiating and digesting the powder in pepsin and HCl for 72 h, and neutralization to a physiological pH, salinity and temperature. (B) DNA quantification in fresh (immediately after organ harvest) and decellularized piglet mucosa. Mean±S.D. (n=3). Student t-test p-value<0.05. Asterisk denotes significance. (C) Histological sections of fixed ECM gel drops stained with Picrosirius Red, Verhoeff's and Alcian Blue for collagen, elastin and glycosaminoglycans, respectively. Scale bar 200 μm. (D) Quantification of ECM proteins: collagen, elastin and GAG. Mean±S.D. (n=3). Student t-test p-value<0.05. Asterisk denotes significance. (E) Analysis of the collagen types in ECM gel and Matrigel by staining for collagen I, Ill and IV. Scale bar 100 μm. (F) Scanning electron microscopy (SEM) images of the ECM gel displaying the interconnected fibrous network. Scale bars 1 μm. (G) Spectrophotometry used to assess the turbidity of the samples during gelation. Mean±S.D. (n=3). Student t-test p-value<0.05 (h-i) Oscillatory rheology provides a rheological profiles of various concentrations of the ECM gel and Matrigel, for both (H) storage modulus and (I) loss modulus. (J) Elastic modulus measured by nanoindentation of 6 mg/mL ECM gel vs. Matrigel in 30 μL drops. Mean±S.D. (n=3). Student t-test p-value<0.05. (K) Images of the laboratory procedure for piglet small intestine mucosa/submucosa decellularization. (L) Sus scrofa small intestine sections pre- and post-decellularization staining. Hematoxylin/eosin, Picrosirius Red, Verhoeff's and Alcian Blue for cell nuclei, collagen, elastin and glycosaminoglycans, respectively. The images show complete removal of antigenic cellular material, and high preservation quality of extracellular matrix proteins after the process of decellularization. Scale bars 250 μm. (M) Spectrophotometry graphs representing sample gelation kinetics for 10 mg/mL ECM gels freshly prepared at 4° C., stored at −20° C., and stored at room temperature for 1 month. No statistically significant difference in the Tlag is observed between the freshly prepared and the −20° C. stored. RT stored gel fails gelation as no sigmoidal curve is observed. Values are mean of 2 biological replicates, 7 technical replicates each. Student t-test p-value≤0.05.

These figures are discussed in example 1.

FIG. 2—ECM proteomic analysis. (A) Protein abundance range, with 619 (on 1617 total) proteins mapped to GO-CC:0070062˜extracellular exosomes highlighted. Yellow-shaded area represents the range covering 90% of total protein abundance. Collagens analyzed in FIG. 1 are also highlighted. (B) Relative abundance of selected ECM proteins. (C) Hierarchical clustering analysis of mass spectrometry native human tissue data from a draft map of the human proteome, conducted for proteins in our data mapped to GO-CC:0031012˜ECM. Four main clusters are identified whose color-coded tissues are reported on the right. A small group of proteins especially expressed in cluster 3 is highlighted. A fully detailed version of this heatmap is reported in FIGS. 2E-2G. (D) Principal component analysis (PCA) of data from native human tissue reported in C, and of data generated in this study. Tissues and samples having endodermal origin are also highlighted. Mean±SEM (n=3, with 3 technical replicates each). (E) Metabolomics screening. List of compounds found in all 3 different batches of SI ECM pre-gel analyzed. (F) Number of unique peptides and protein sequence coverage for the ECM proteins shown in FIG. 2B. (G) Full resolution results of the hierarchical clustering analysis reported in FIG. 2C, image has been 90-degree rotated for clarity.

These figures are discussed in example 2.

FIGS. 3-3D culture of endodermal stem cells in ECM hydrogel and Matrigel. (A) 3D culture of human pediatric gastric enteroids in ECM gel. (B) Planes of whole-mount immunofluorescence of 7-days human pediatric gastric organoids showing both epithelial (zonula occludens-1, epithelial cadherin and actin) and gastric (ezrin and mucin-5AC) markers. Scale bar 50 μm. (C) Culture of human liver ductal and human hepatic organoids in ECM gel, and both in BME and Matrigel as controls. Scale bar 500 μm. (D) Hepatic organoid viability assessed through Cell Titer-Glo assay shows no significant difference among the three conditions. (E) Bright field and H&E images of the mouse intestinal enteroids show morphologically similar cells for both the ECM gel and Matrigel. Scale bars 100 μm. (F) Immunofluorescence analysis of sections of mouse SI organoids in ECM gel and Matrigel control, showing epithelial cadherin staining and proliferation marker Ki-67+. Scale bar 50 μm. (G) Immunohistochemical staining of mouse intestinal enteroids in ECM gel and Matrigel. Cells in ECM show comparable expression to control of intestinal differentiation markers such as lysozyme, mucin-2 and villin. Scale bars 25 μm. (H) Forming mouse intestinal organoids per field of view at day 4 of culture in ECM gel and matrigel show no significant difference over 2 passages. Mean±S.D (n≥12). (I) Live/Dead assay of human pediatric small intestinal organoids cultured in ECM gel and Matrigel. Calcein-AM shows live cells. Ethidium homodimer-1 shows dead cells. Scale bar 50 μm. (J) Quantification of vital cells from Live/Dead assay. Mean±S.D (n≥10). (K) Morphology of 8 consecutive passages over a period of 2 months of human pediatric small intestinal organoids in ECM gels. Scale bar 300 μm. (L) Analyses of 4 consecutive passages of human pediatric small intestinal organoids diameters at day 3 of culture, showing comparable dimensions in ECM gel and Matrigel control. Mean±S.D (n>30). (M) Bright field of human fetal small intestinal enteroids in ECM gel. Scale bar 200 μm. (N) Z-Planes of whole-mount immunofluorescence of human fetal small intestinal organoids showing crypt stem cell marker olfactomedin-4, crypt Paneth cell marker lysozyme, villi enterocyte marker keratin-20 and actin staining. Scale bar 100 μm. (0) Analyses of 3 consecutive passages of human fetal small intestinal organoids diameters at day 3 of culture, showing comparable dimensions in ECM gel and Matrigel control. Mean±S.D (n>30). (P) Single-cell colony formation capacity assessed over 3 days in disaggregated human fetal small intestinal organoids in ECM gel and matrigel. Scale bar 25 μm. (Q) Direct derivation of human gastric organoids, and human small intestinal organoids, from pediatric donor biopsies in 4 mg/mL small intestinal ECM gel and Matrigel control. Scale bars 200 μm. (R) Immunofluorescence staining of tissue and gel cryosections. Nuclei in blue, FITC-conjugated B4 isolectin (BSI-B4; Griffonia (Bandeiraea) simplicifolia) in green, and anti-alpha-Gal antibody (M86) in red. Immunofluorescence shows presence of alpha-gal antigen in fresh piglet small intestinal tissue, residual antigen presence in decellularized tissue, absence in 6 different batches of piglet ECM gels. Scale bars 100 μm. Co-polymerization of the ECM-derived hydrogel with photo-crosslinkable polyacrylamide to design a flat hydrogel with tunable properties. (S) Scanning electron microscopy (SEM) images of the ECM-PA co-polymer hydrogel displaying the homogenous distribution of the ECM fibers on the surface of the polymer. Scale bars 10 μm and 1 μm. (T) Nanoindentation characterization. This graph represents the Young's modulus of different hydrogel with growing concentration of polyacrylamide compared to ECM, showing the possibility to tune the stiffness properties of the co-polymer. (U) Mouse and human small intestinal organoids disaggregated to single cells and plated as monolayer on the ECM-PA hydrogel. The cells show adhesion and proliferation until confluence. Scale bar 100 μm. (V) Immunofluorescence staining showing epithelial colony organization. Scale bars 50 μm.

These figures are discussed in example 3.

FIG. 4—Transcriptomic analysis results of different ECM organoids. (A-E) Pediatric SI organoids. (A) PCA analysis. (B) Number of DEGs up- and down-regulated in ECM compared to Matrigel for different absolute log-fold change ratios. (C) Expression of genes selected for their involvement in the indicated processes. Mean±S.D. (n=4). Black asterisks indicate DEGs. (D) Heat map of expression of core matrisome DEGs ordered according to hierarchical clustering. FIG. 3)S reports the corresponding analysis for matrisome-associated transcripts. (E) Selected GO categories enriched in DEGs between ECM and Matrigel involved in the interaction of cells with the extracellular space. (F) Real-time PCR analysis of SI transcripts. Mean±SEM (n=4). Student t-test p-value<0.05. (G) Principal component analysis (PCA) plot on human ductal liver organoids cultured in ECM gel vs BME. (H) Heat map of top 20 upregulated and top 20 downregulated genes ECM gel vs BME ductal organoids. (I) Ductal liver transcripts plot comparison in ECM gel vs. BME. Mean±S.D. (n=4). Black asterisks indicate DEGs. (J) Principal component analysis (PCA) plot on human fetal hepatocyte organoids cultured in ECM gel vs. BME. (K) Heat map of top 20 upregulated and top 20 downregulated genes ECM gel vs BME hepatocyte organoids. (L) Hepatic transcripts plot comparison in ECM gel vs. BME. Mean±S.D. (n=4). (M) Comparison of ALB expression in ductal and hepatocyte organoids by ELISA assay. Mean±S.D. (n=8). Red dots on the bar charts represent single data points throughout the figure. RNA-seq analysis of human organoids cultured in ECM gel vs Matrigel. (N-O) Results of human pediatric small intestinal organoids. (N) Hierarchical clustering of ECM-associated DEGs. (O) Results of over-representation analysis of DEGs within the following GO categories: GO-BP (top), GO-CC (middle), and GO-MF (bottom). Similar categories are clustered according to kappa score and indicated by different colors. Larger circle size indicates higher significance. Benjamini-Hochberg-corrected p-values from right-sided hypergeometric test. GO-BP corrected p-value<0.001, GO-CC and GO-MF corrected p-values<0.05. (P) Cluster map of human ductal liver organoids cultured in ECM gel vs Matrigel. (Q) Cluster map of human fetal hepatic organoids cultured in ECM gel vs Matrigel.

These figures are discussed in example 4.

FIG. 5—In vivo delivery of ECM cultured organoids (A) 3D culture of human fetal pancreatic ducts in ECM gel and Matrigel. Bright field and H&E images of the human fetal pancreatic enteroids show morphologically similar cells for both the ECM gel and control. Scale bars 100 μm. (B) Immunofluorescence analysis of sections of fetal pancreas organoids in ECM gel and Matrigel, showing comparable expression to control of mucin-1A, epithelial cadherin, together with insulin promoter factor 1 and cytokeratin-19. Scale bar 50 μm. (C) Analyses of 3 consecutive passages of human fetal pancreatic organoid diameters at day 6 of culture, showing comparable dimensions in ECM gel and Matrigel control. Mean±S.D (n>30). (D) Forming pancreatic ducts per field of view at day 6 of culture in ECM gel and Matrigel. Mean±S.D (n≥12). (E) Evaluation of the ECM gel vascularization potential through Chick Chorioallantoic Membrane (CAM) Assay. (F) Quantification of the number of blood vessels directed towards the gel on the CAM shows no significant difference in the angiogenic potential between the ECM gel and the Matrigel. Mean±S.D (n≥3). (G) ECM gel and Matrigel are circled in blue on the CAM. Scale bar 1 mm. (H) H&E staining of the CAM showing a comparable interface between the ECM gel and Matrigel. Scale bar 250 μm. (I) Mouse subcutaneous transplantation of human fetal pancreatic ducts in ECM gels. Recovery of silicon rings from mouse back with ECM gels (blue arrow) after 2.5 weeks (above—scale bar 5 mm). H&E staining of pancreatic ducts showing good morphology after in vivo transplantation in ECM gel (below—scale bar 100 μm). (J) Immunofluorescence staining of human fetal ducts in ECM gel after 2.5 weeks in vivo, showing high expression of pancreatic markers mucin-1 (with polarized luminal localization), epithelial cadherin, insulin promoter factor 1 and cytokeratin-19. Scale bars 25 μm. (K) Immunofluorescence staining of Matrigel control human fetal ducts in Matrigel after 2.5 weeks in vivo. Scale bars 100 μm. (L) Mouse subcutaneous transplantation of mouse LGR5-DTR-EGFP small intestinal organoids in ECM gels. Recovery of silicon rings from mouse back with ECM gels (blue arrow) after 4 weeks (above—scale bar 1 mm). Bright field image of ECM gel with intestinal organoids inside (below—scale bar 200 μm). (M) Immunofluorescence staining of mouse LGR5-DTR-EGFP small intestinal organoids in ECM gel after 4 weeks in vivo, showing high expression of crypt/stem markers anti-GFP-LGR5, olfactomedin-4 and lysozyme, together with villi/differentiation markers cytokeratin-20, L-type fatty acid binding protein and mucin-2. Scale bars 100 μm.(N)-(P) Two months mouse subcutaneous transplantation of human fetal pancreatic organoids in ECM gels and Matrigel. (N) Recovery of silicon rings from mouse back with ECM gels after 2 months. Scale bar 5 mm. (O) H&E staining of pancreatic ducts showing comparable morphology after in vivo transplantation in ECM gel and control Matrigel. Scale bar 500 μm. (P) Immunofluorescence staining of human fetal ducts in ECM gel and Matrigel after 2 months in vivo, showing high expression of pancreatic markers insulin promoter factor 1, epithelial cadherin and cytokeratin-19. Scale bar 100 μm.

These figures are discussed in example 5.

### EXAMPLES

The following examples are provided so as to provide those of ordinary skill in the art with a complete disclosure and description of how to make the compositions of the invention and how to practice the methods of the invention and are not intended to limit the scope of what the inventor regards as his invention. Efforts have been made to ensure accuracy with respect to numbers used (e.g. amounts, temperature, etc.), but some experimental errors and deviation should be accounted for. Unless indicated otherwise, temperature is in degrees C., chemical reactions were performed at atmospheric pressure or transmembrane pressure, as indicated, the term “ambient temperature” refers to approximately 25° C. and “ambient pressure” refers to atmospheric pressure. The invention will be further clarified by the following examples which are intended to be exemplary of the invention.

### Example 1—Gelation and Characterization of ECM-Derived Intestinal Hydrogel

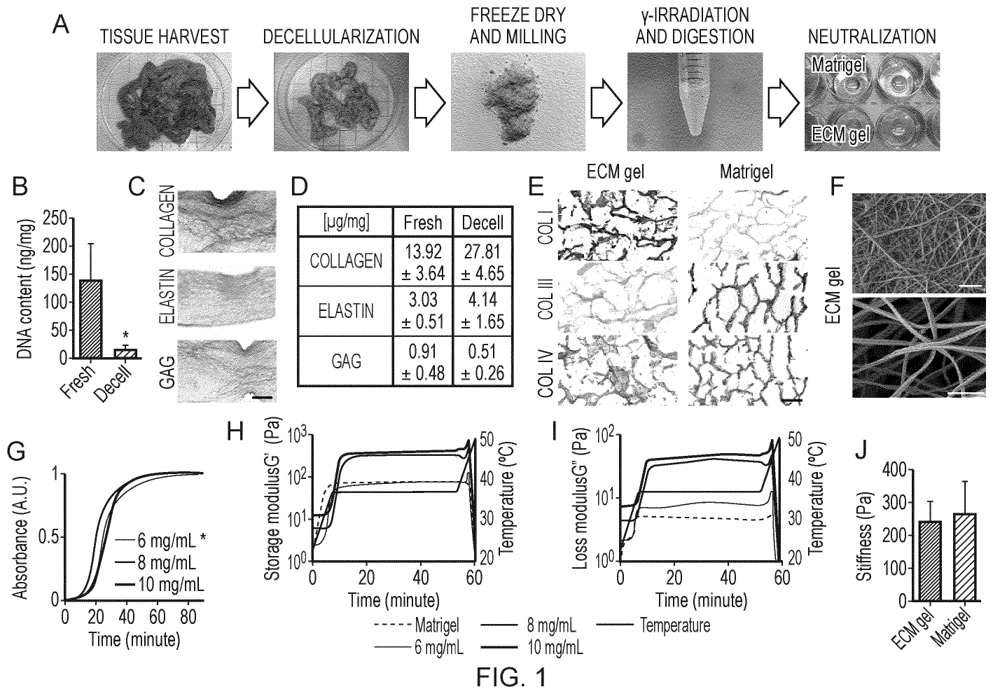

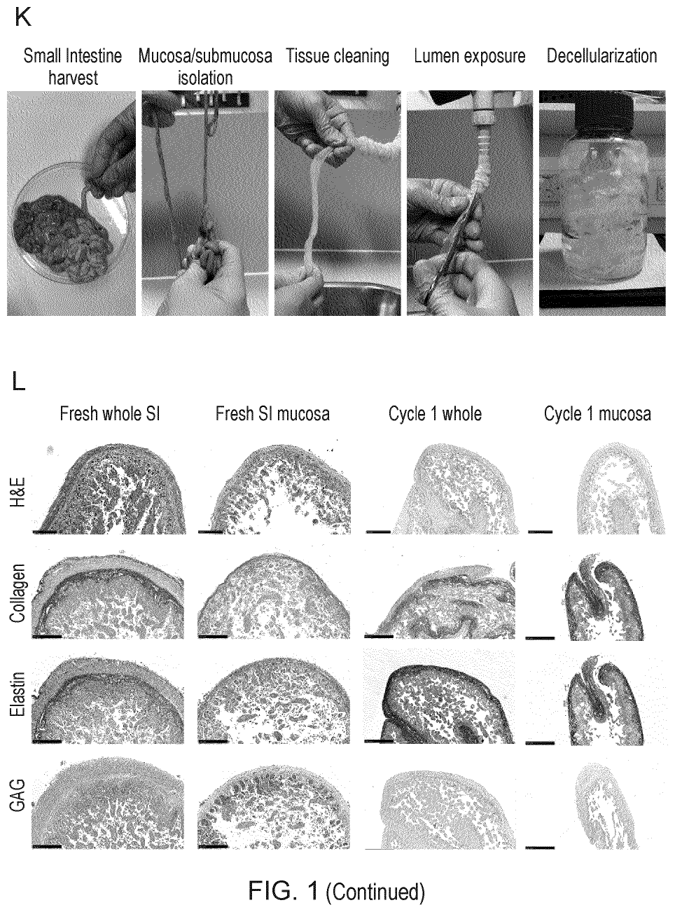

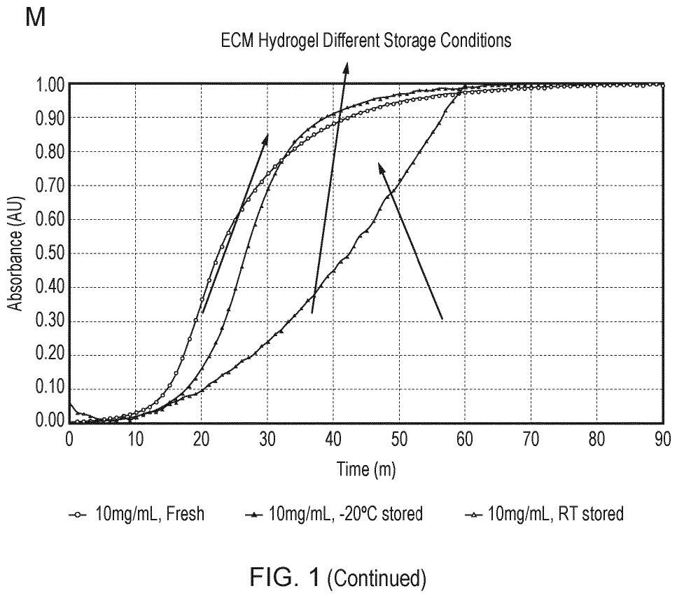

To optimize a GMP-compatible process for ECM gel, a 5 step protocol was designed which includes (i) tissue harvesting; (ii) decellularization; (iii) freeze dry and milling; (iv) gamma-irradiation and digestion; and (v) neutralization (FIG. 1A) based on modification of previously reported protocols¹⁸⁻²⁰ (FIG. 1A, FIG. 1K). Only one cycle of the detergent-enzymatic treatment (DET) facilitated nuclei removal and significant DNA decrease (FIG. 1B) in a porcine intestinal scaffold. This short protocol minimized morphological tissue alteration compared to other decellularization protocols, as previously reported²⁰ (FIG. 1L).

It was first verified that ECM powder derived from porcine intestinal tissue successfully formed a hydrogel when following a gelation protocol. ECM powder was digested in pepsin and HCl to form a pre-gel, re-equilibrated to neutral pH and exposed to physiological temperature. The SI ECM decellularization and gelation efficiently preserved the relevant extracellular matrix components including collagens, elastin and still contained glycosaminoglycans (FIG. 1C-D). When compared to standard 3D culture systems such as Matrigel, collagen I, Ill and IV showed at least comparable signals (FIG. 1E). Solubilization of ECM by pepsin digestion was performed to preserve the ultrastructure of the collagen fibers, based on the fact that pepsin cleaves collagens in locations where the three alpha-chains are not interacting to form a stable triple-helical structure²¹. To further investigate the structure of the ECM hydrogels, scanning electron microscopy was performed and showed the detailed interwoven network of collagen fibers (FIG. 1F).

Rheological and mechanical properties of 3D environment are relevant to organoids culture. By spectrophotometry the turbidimetric gelation kinetics of the hydrogels were assessed (FIG. 1G). All the analyzed ECM hydrogel concentrations (6, 8 and 10 mg/mL) formed a sigmoidal curve indicating gelation, reaching the 90% of gelation in ≈30 min for all 3 conditions. T_{tag} time was significantly shorter at ECM gel concentration of 6 mg/mL, compared to 8 or 10 mg/mL (see Table 1).

TABLE 1
Gelation characterization of different ECM concentrations

| | | | Gelation Half-time |
|---|---|---|---|
| | Lag Period (T_{lag}) | Gelation Rate (S) | (T_{1/2}) |
| ECM gel | (min, ±SD) | (AU/min, ±SD) | (minutes, ±SD) |
| 10 mg/ml | 16.39 ± 0.63 | 0.047 ± 6.33 × 10⁻⁴ | 26.92 ± 0.78 |
| 8 mg/ml | 15.96 ± 0.82 | 0.048 ± 7.59 × 10⁻⁴ | 26.30 ± 0.59 |
| 6 mg/ml | 12.79 ± 1.37* | 0.059 ± 2.66 × 10⁻³ | 21.25 ± 0.83 |

However, the ECM powder digested solution (pre-gel) preferably needed to be freshly prepared and kept at +4° C., or stored at −20° C., because, in these conditions, room temperature (1 month) stored pre-gel failed gelation as no sigmoidal curve was observed (FIG. 1M).

Rheological characteristics of different ECM gel concentrations and Matrigel were assessed using temperature ramping oscillatory rheology (FIG. 1H-I). All concentrations of the ECM gel and Matrigel exhibited gel-like properties when exposed to 37° C. temperature, with the storage modulus (G′) higher than the loss modulus (G″). At approximately 45° C. all gels experienced a drop in their storage modulus, indicating a melting point for the gels including Matrigel. ECM gel at 6 mg/mL exhibited a similar rheological profile to the Matrigel, in terms of storage and loss modulus, and was preferentially used for cell culture purposes. Consistently, elastic modulus analyzed by nanoindentation showed how 6 mg/mL ECM gels and Matrigel displayed a comparable elastic modulus (FIG. 1J).

### Example 2—Intestinal ECM Hydrogel Proteomic Profiling

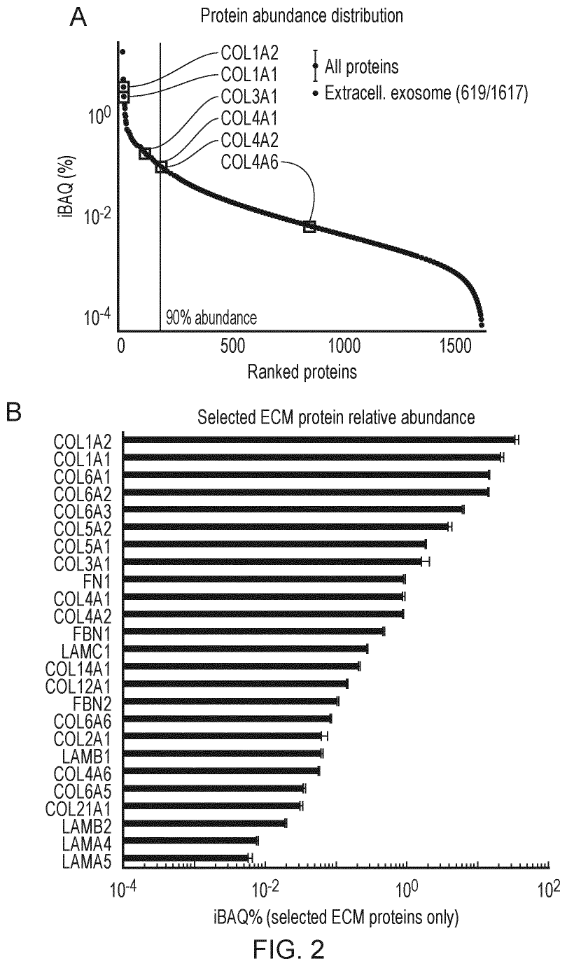

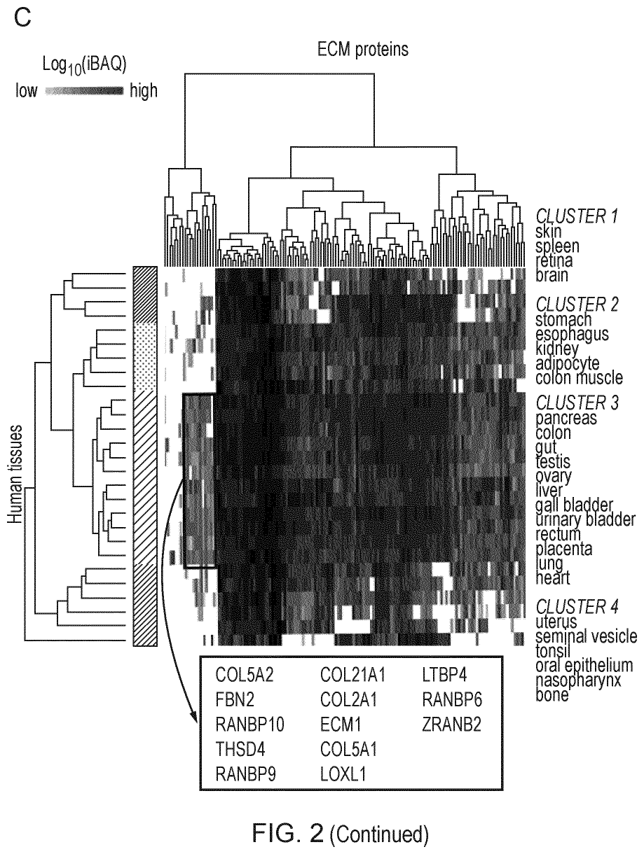

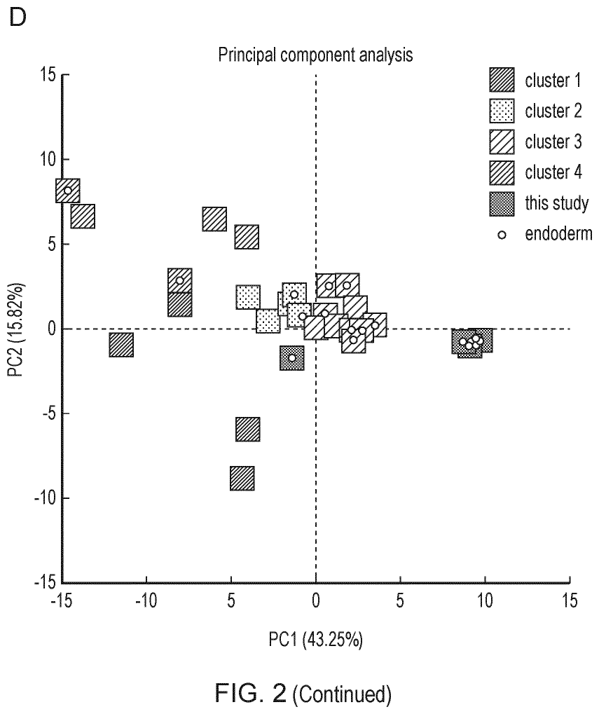

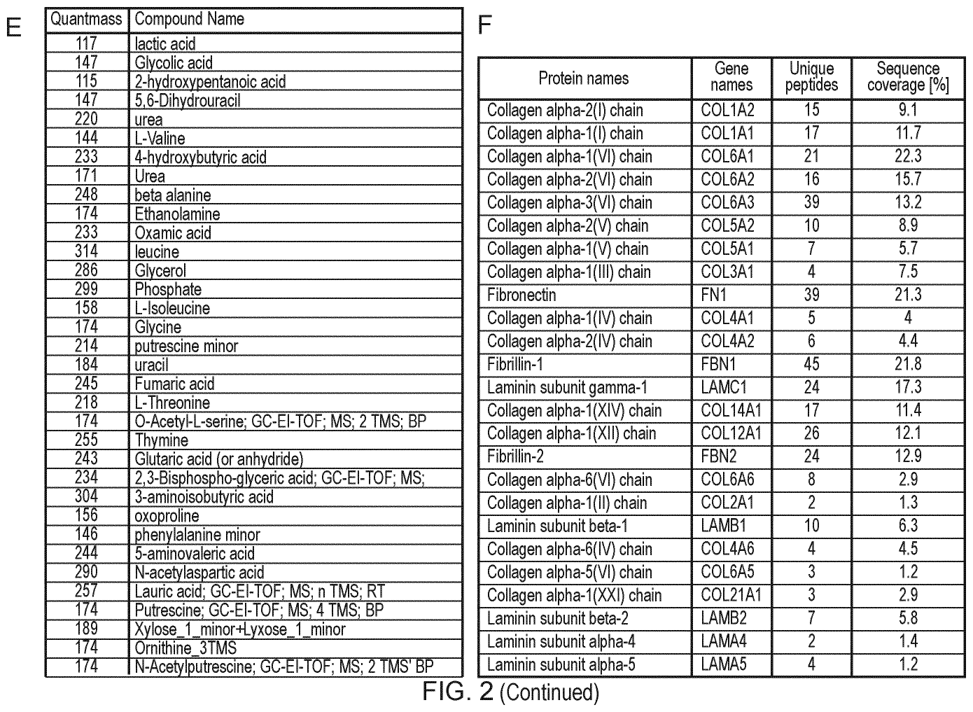

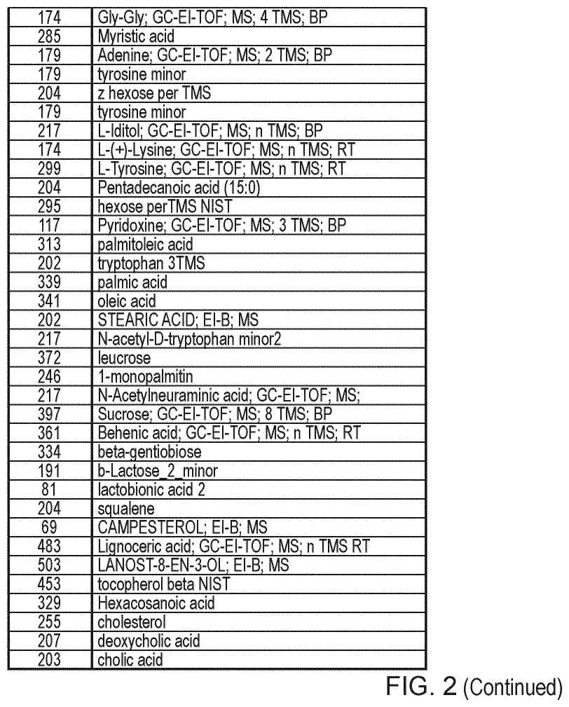

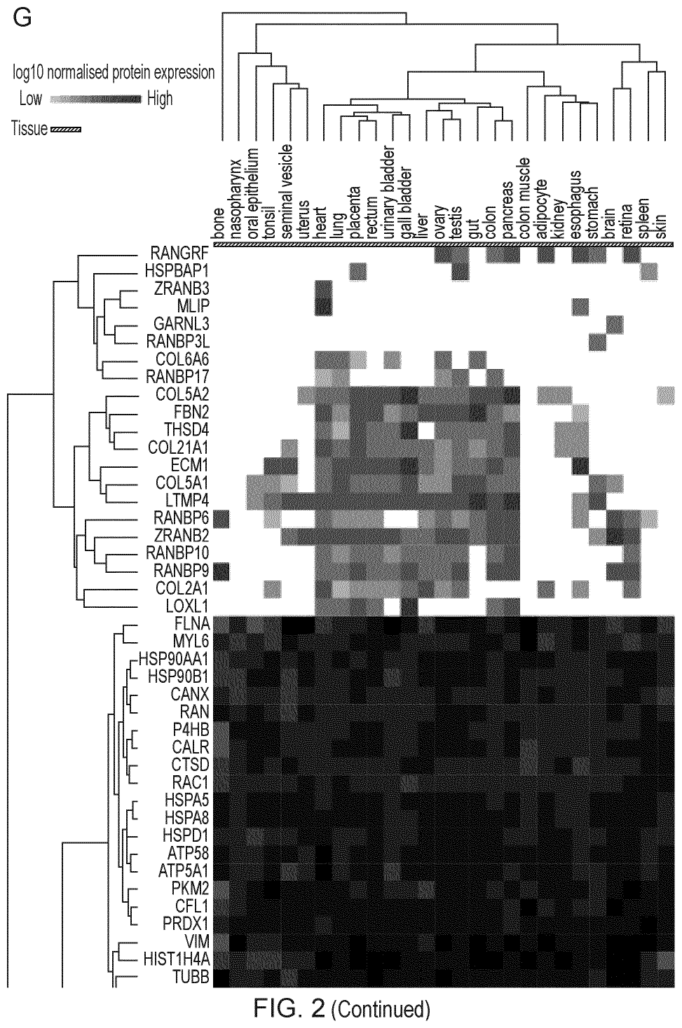

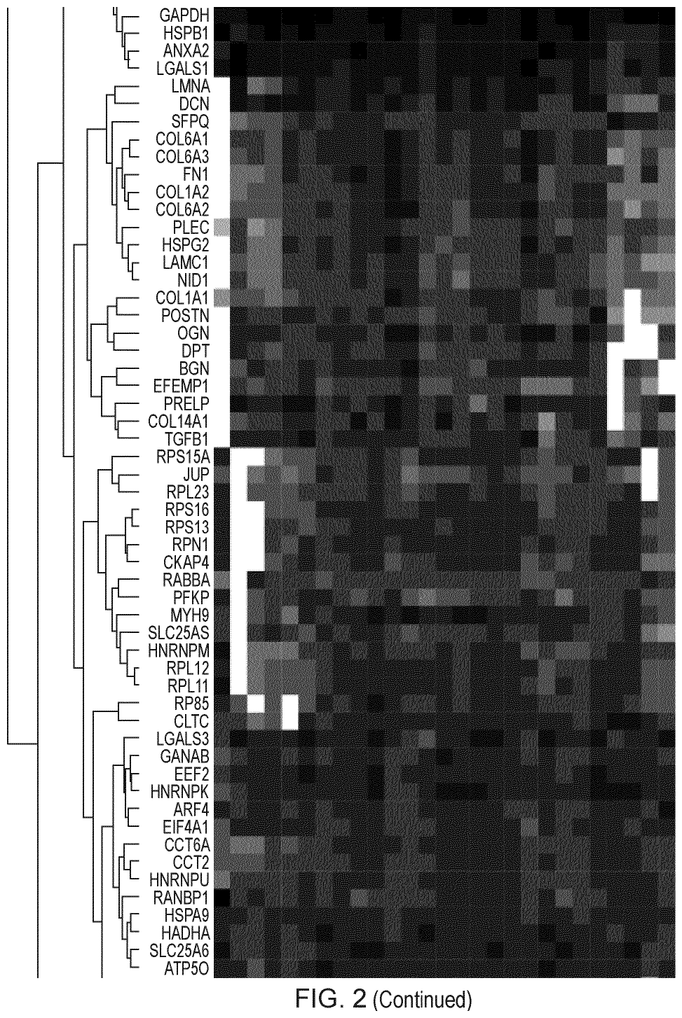

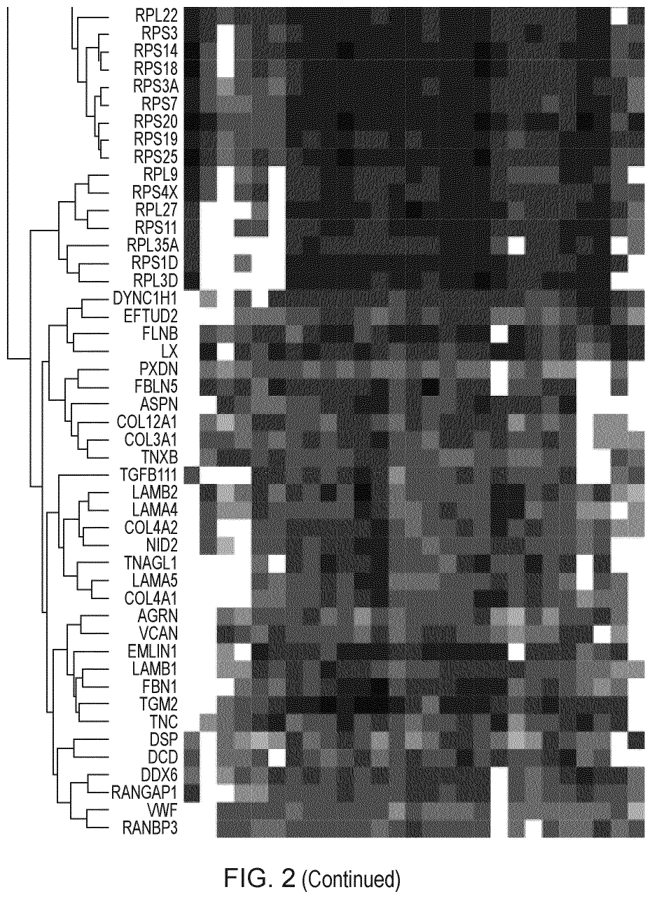

To characterize the ECM composition in terms of residual proteomic content after decellularization, a mass spectrometry analysis was performed on the powder form, before pepsin digestion¹¹. More than 1600 proteins were identified, of which ˜130 were recognized as derived from ECM and 619 from extracellular exosomes (FIG. 2A), confirming the dual role of ECM as a supportive structure and as a storage of adsorbed soluble signals. Exosomal proteins are most significantly over-represented in pathways related to translation and cell adhesion. The abundance of RNA-binding proteins within exosomes is well known and their preservation could play a role in intercellular signaling during differentiation²². A separate set of metabolomics experiments were performed to further analyze the residual compounds found in the pre-gel, after pepsin treatment. Expected compounds such as fatty acids residuals and sub-products of the protein metabolism were found in all three different batches analyzed (FIG. 2E).

Despite the high diversity of identified proteins, most of them contributed for less than 1% of the total amount. Multiple collagen types were among the most abundant, mostly fibrillar (type 1, 2, 3, 5, 6), but also fibril-associated (type 12, 14, 21) and sheet forming (type 4) (FIG. 2B, FIG. 2F).

To verify if the retained complex protein composition of the ECM gels showed similarities with specific human tissues, the ECM proteins quantified in the data were searched on a publicly available map of the human proteome (FIG. 2C). This analysis was restricted to more relevant tissues for regenerative medicine applications. The protein set given by the ECM proteins in the data (˜130 proteins) was sufficient to identify a cluster of tissues that show a similar proteomic profile and comprise multiple endoderm-derived tissues, including gut, liver and pancreas (cluster 3 in FIG. 2C). A group of proteins that are almost exclusively expressed within this cluster (and in our samples) was identified. It includes not only structural constituents of the ECM, such as collagens, but also proteins responsible for cross-linking collagen fibrils and forming elastic fibers (LOXL1, FBN2). A full resolution panel of results of the hierarchical clustering analysis is reported in FIG. 2G.

The similarity between the ECM protein composition of the decellularized matrices and the above tissues was also quantitatively investigated by principal component analysis, which showed a higher similarity of the ECM gels composition with tissues of endodermal origin (FIG. 2D). The decellularization process was able to preserve protein composition features that are not only characterizing the native tissue of the ECM gels, but also shared within a group of similar developmental origin tissues.

### Example 3—ECM Hydrogels Support Both Mouse and Human Organoids Cultures

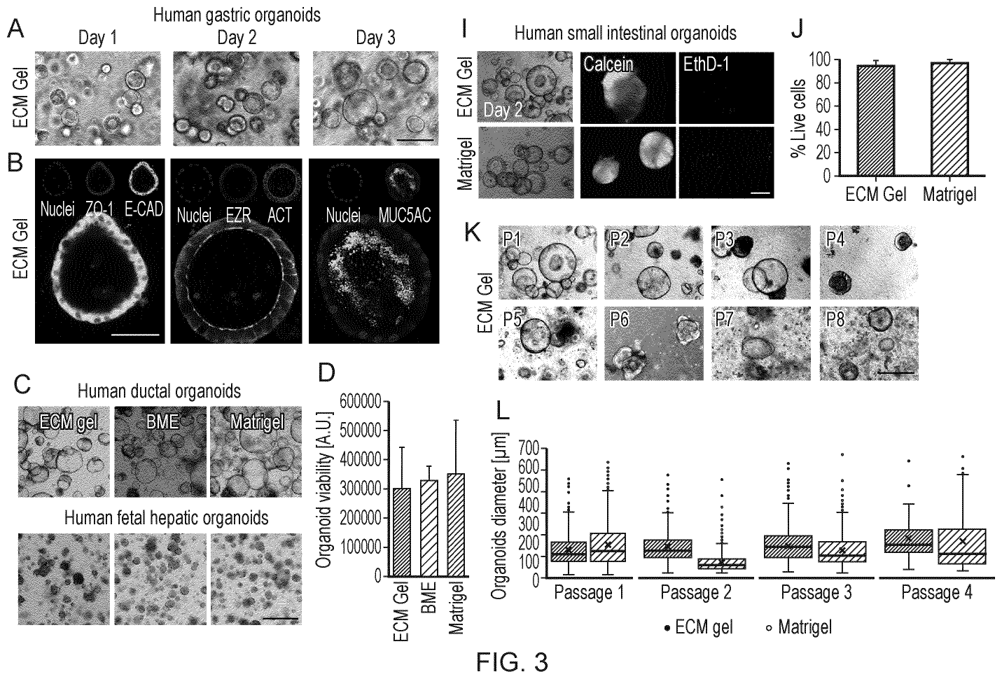

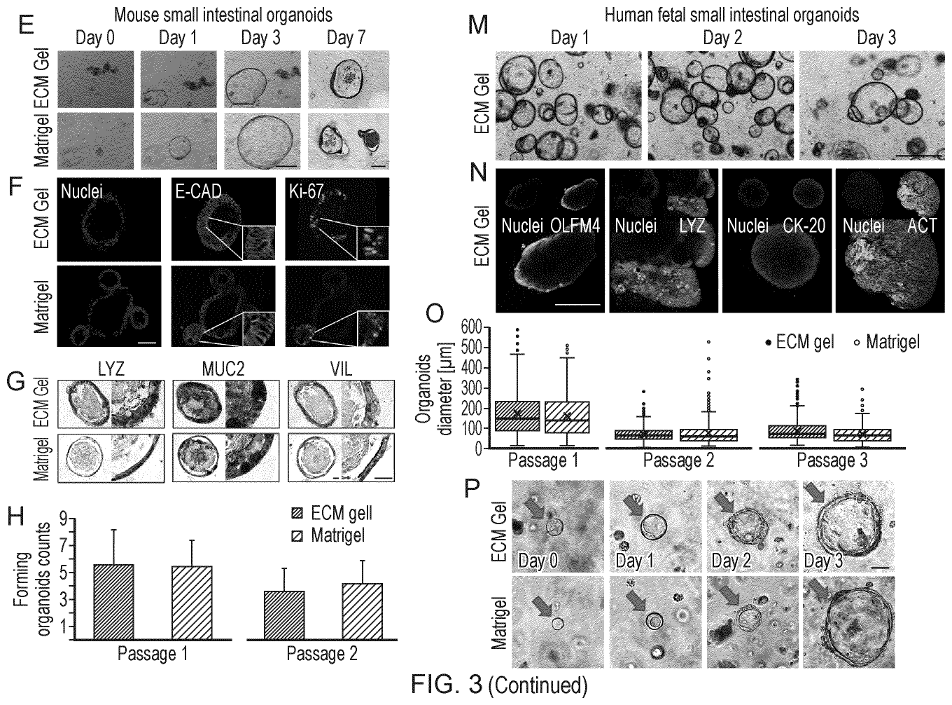

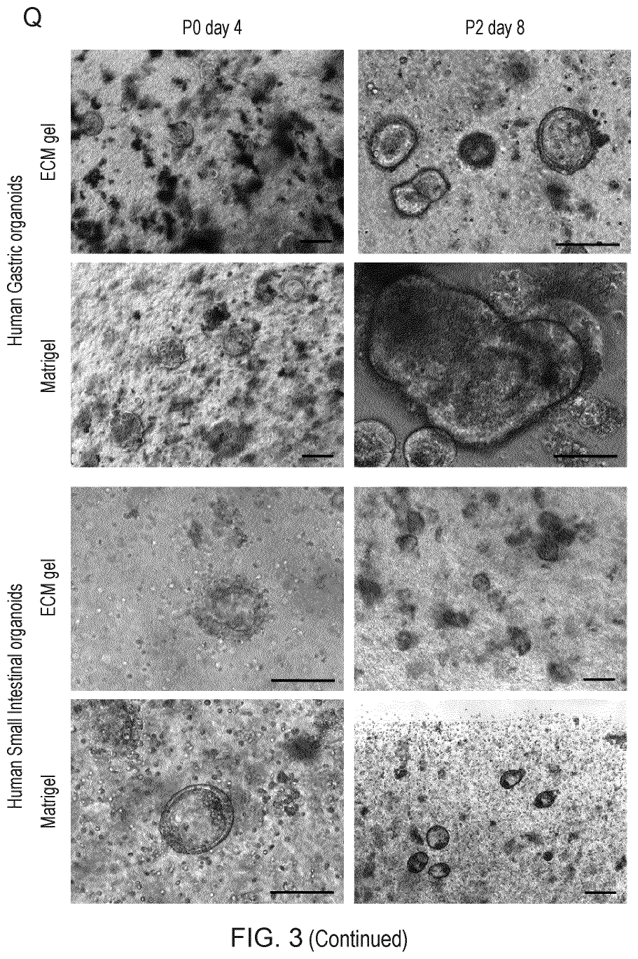

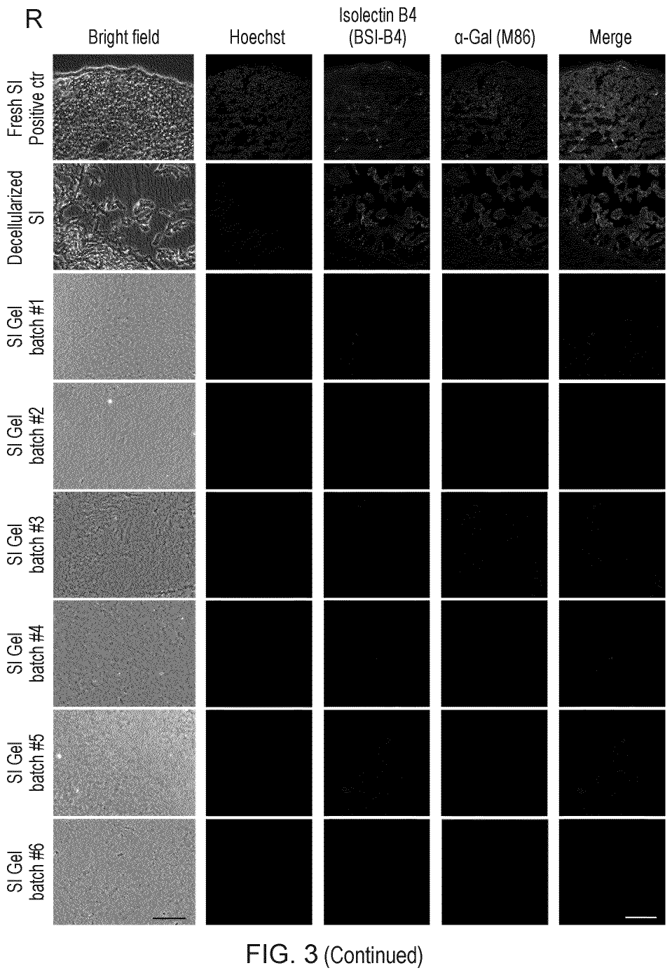

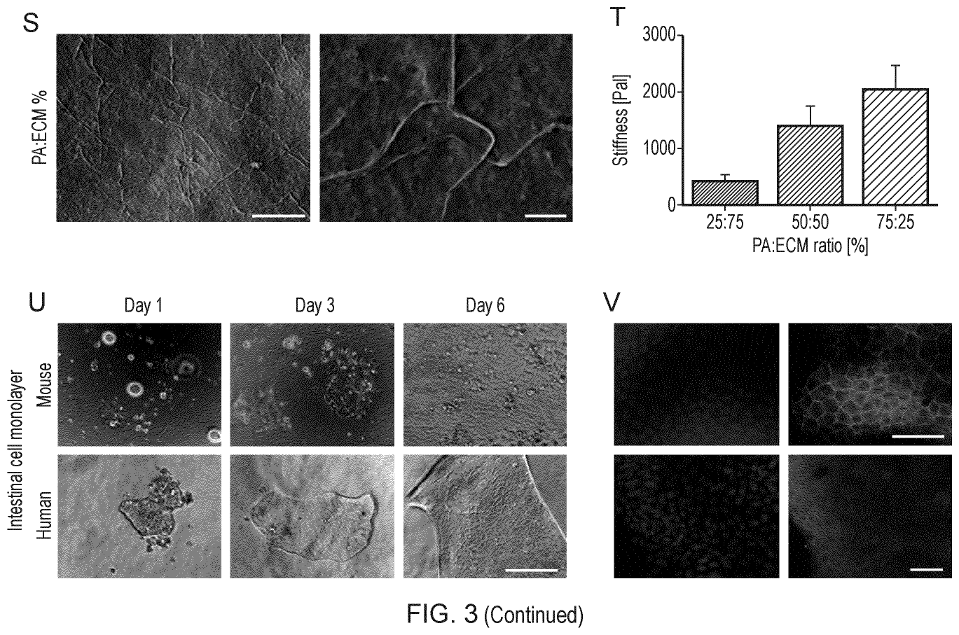

The possibility of ECM gels to host different endoderm-derived organoids cultures was then demonstrated. Extensive analysis was performed on both human and mouse organoid cultures, from different organs. First, human organoids of gastric origin showed high level of adaptation to the small intestinal ECM gel. The pediatric stomach enteroids maintained the expression of both epithelial (zonula occludens-1, epithelial cadherin and f-actin) and gastric (ezrin and mucin-5AC) markers after 7 days of culture (FIG. 3A-B).

Different endodermal organoids such as human ductal (cholangiocytes) and human fetal hepatic (hepatocytes) organoids from different donors were also explored, and the morphology with two different standard controls such as BME and Matrigel (FIG. 3C)^{23,24} was compared. No significant difference was observed when culture viability was assessed in the three different conditions (FIG. 3D).

Lgr5+ intestinal stem cells, isolated from the crypts of the mouse small intestine, survived and maintained their phenotype, forming expanding enteroids in the ECM hydrogel over time (FIG. 3E). Proliferating epithelial cells expressing Ki67 were present both in ECM gels and Matrigel (FIG. 3F). Moreover, cells in ECM showed comparable expression to control of intestinal differentiation markers such as mucin-2 and villin, with a higher prevalence of lysozyme (marking Paneth cells) in ECM gel compared to Matrigel cultured organoids (FIG. 3G). The formation of new organoids after split showed no significant difference between gel and Matrigel over the first 2 passages (FIG. 3H).

Importantly, similar features were observed when human pediatric small intestinal organoids were cultured in ECM gels. Live/Dead assay showed a comparable number of live cells to those cultured in Matrigel demonstrating extensive cytocompatibility of ECM hydrogels, with standard viability potential (FIG. 3I-J). ECM gel was shown to also sustain over time human small intestinal cultures, with morphological analyses in 8 consecutive passages, over a time span of 2 months. A decrease in organoid morphological quality was observed in the last 3 passages (FIG. 3K). Nonetheless, during the first four passages, organoids maintained constant dimensions between ECM gel and Matrigel (FIG. 3L).

The possibility of ECM gels to host human organoid cultures of fetal origin was then explored. Small intestinal enteroids showed ideal morphology (FIG. 3M) and high proteomic expression of both crypt (olfactomedin-4 and lysozyme) and villi region (cytokeratin-20/actin) (FIG. 3N). Also in this case organoids maintained comparable dimensions between ECM gel and Matrigel over multiple passages (FIG. 3O). The possibility to split the organoids at single cells, allowing a de novo colony formation after passaging (FIG. 3P) was also confirmed. The possibility to derive organoids from pediatric donors directly in ECM gel, without ever passaging them in Matrigel was also demonstrated. The new formation of organoids from human gastric and human small intestinal biopsies was shown (FIG. 3Q).

To assess the safety of the ECM gel, the residual presence of galactose-alpha-1,3-galactose (alpha-gal) was analysed. The presence of the antigen with immunofluorescence analyses in the non-decellularized small intestinal mucosa, the intact decellularized tissue, and 6 different batches of the piglet SI-derived ECM gel was screened for. It was possible to assess the presence of the porcine antigen both in the fresh and in the whole decellularized tissue, as already reported²⁵. On the other hand, the presence of the antigen in any of the 6 analyzed gels was not observed. These results showed the absence of alpha-gal in the final ECM gel, or the extreme dilution of the epitope that could be detected (FIG. 3R).

It might be important to modulate the physical and biochemical properties of the ECM gel by adding further hydrogel-forming components. By using a synthetic hydrogel system made of poly-acrylamide, a highly homogeneous hydrogel in which the collagen fibers are uniformly interspersed (FIG. 3S) was produced, and the fine-tuning of gel stiffness could be easily achieved (FIG. 3T). Poly-acrylamide, normally a cell repellent, when loaded with ECM gel, allowed cell adhesion and showed the possibility to culture human and mouse small intestinal organoids as monolayers of cells, with an epithelial morphology (FIG. 3U-V).

### Example 4—ECM Gel Culture Human Organoids Transcriptomic Profile

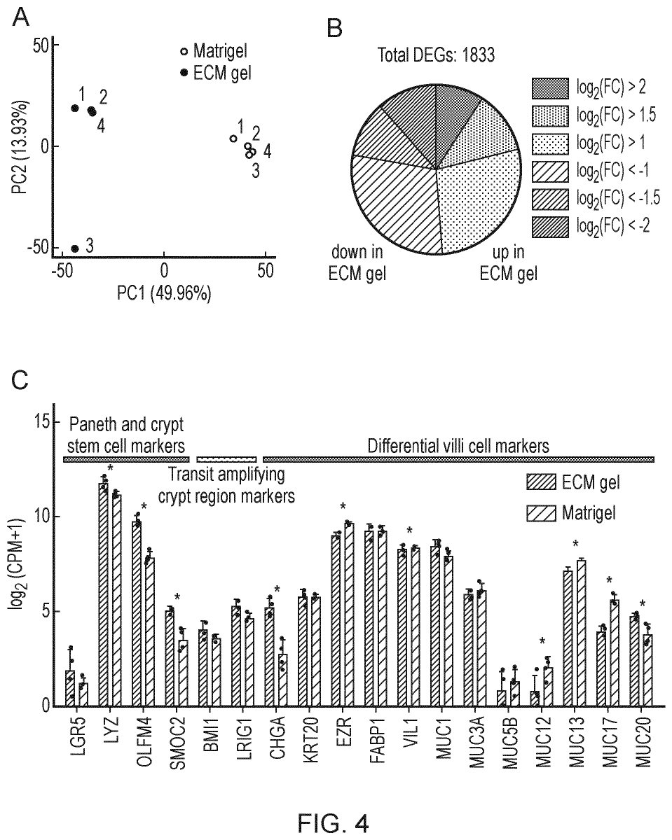

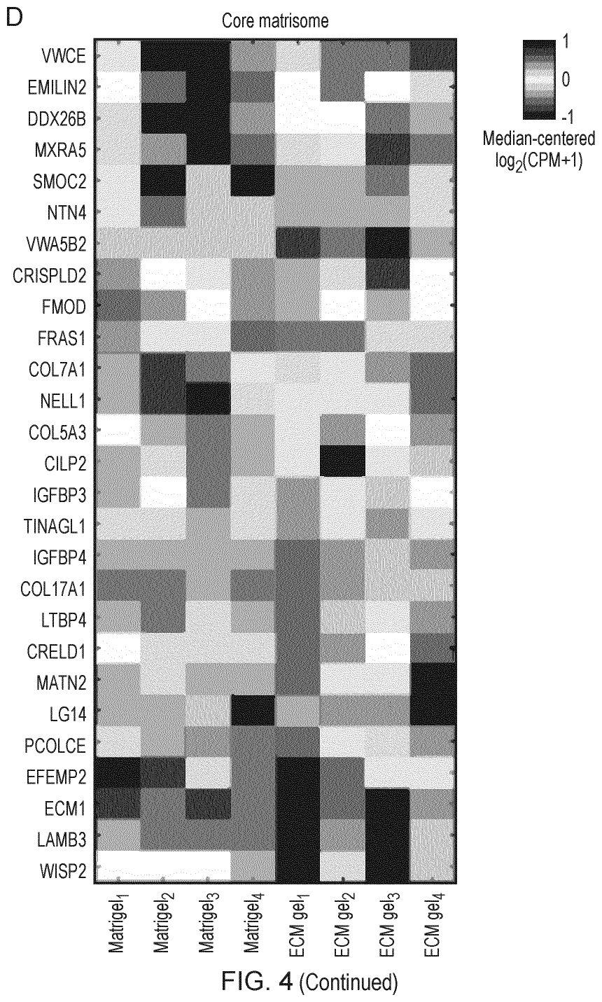

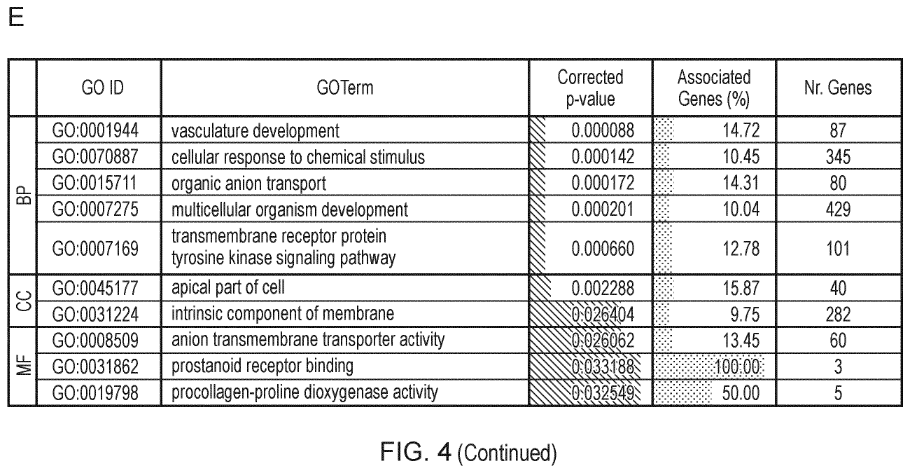

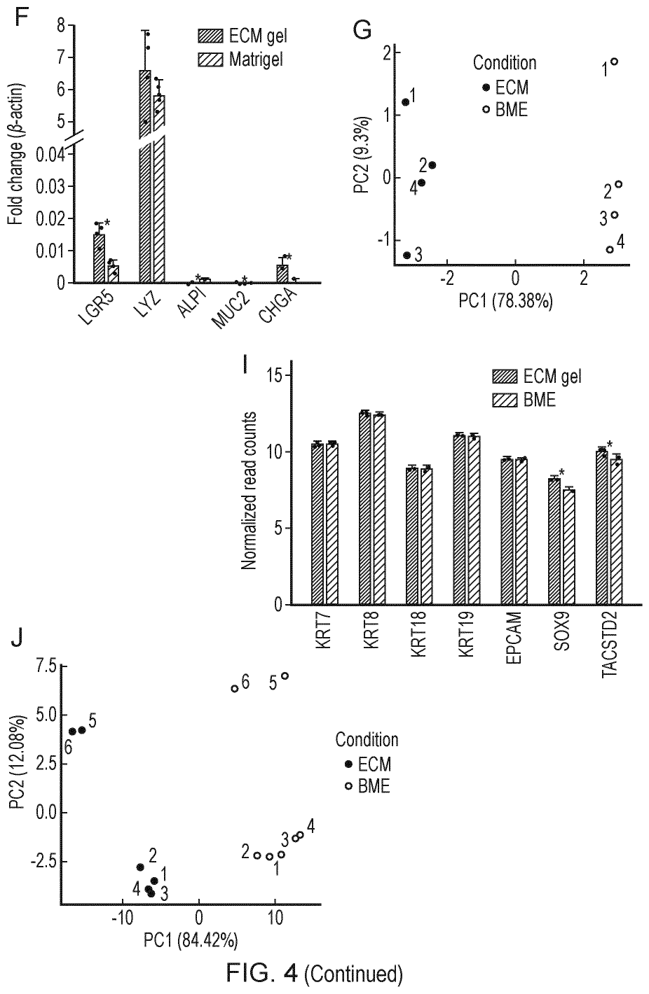

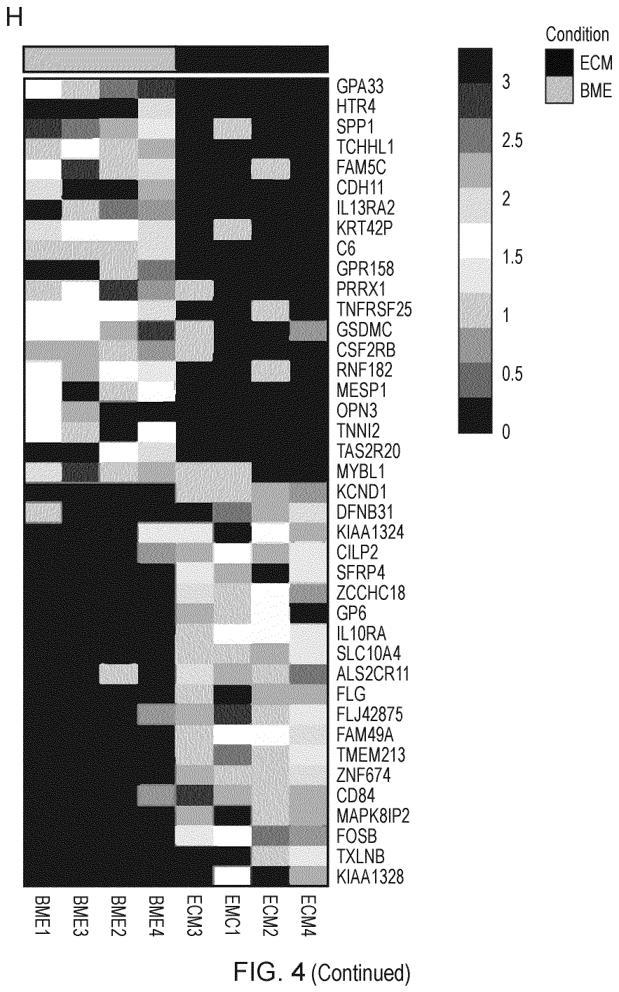

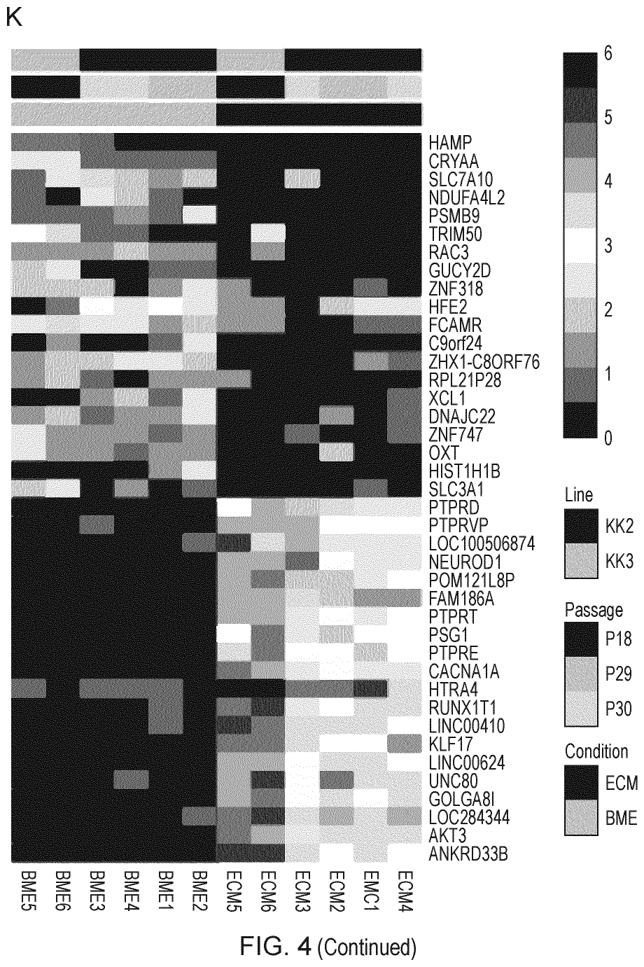

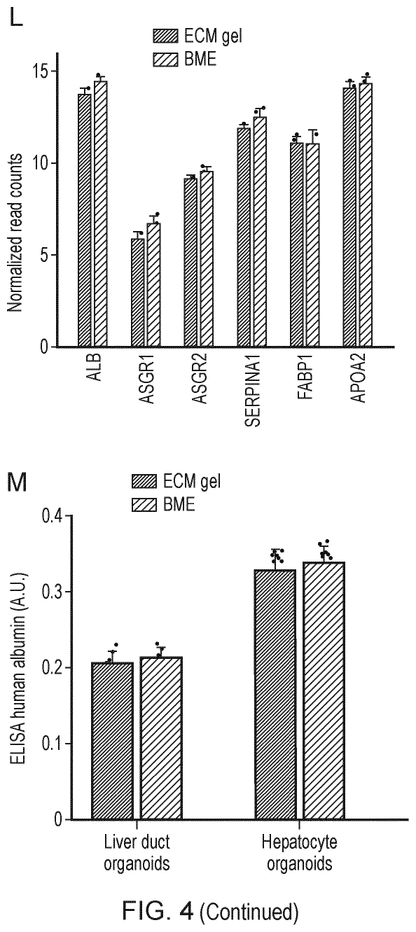

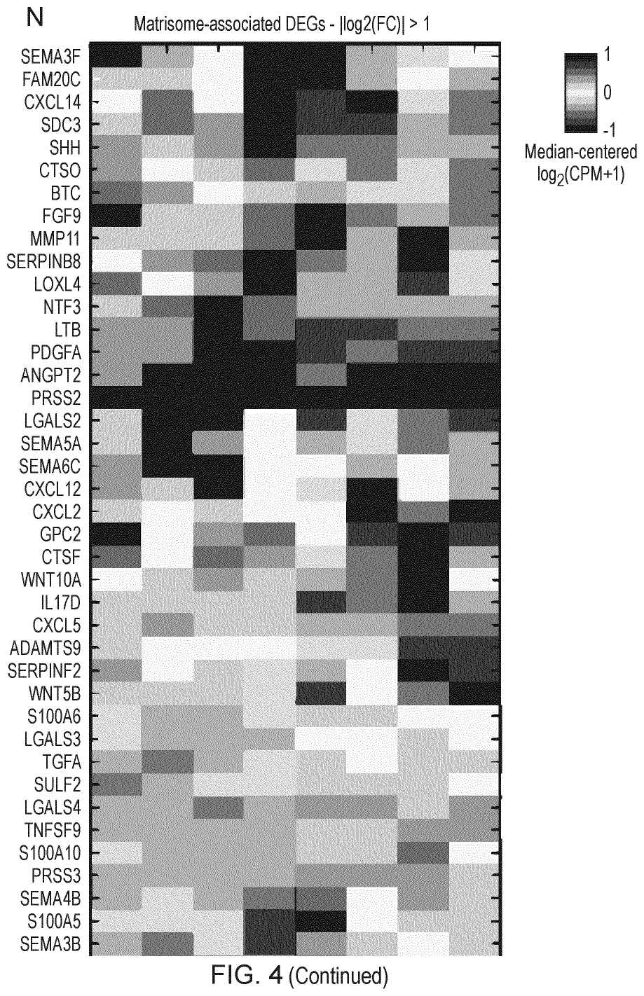

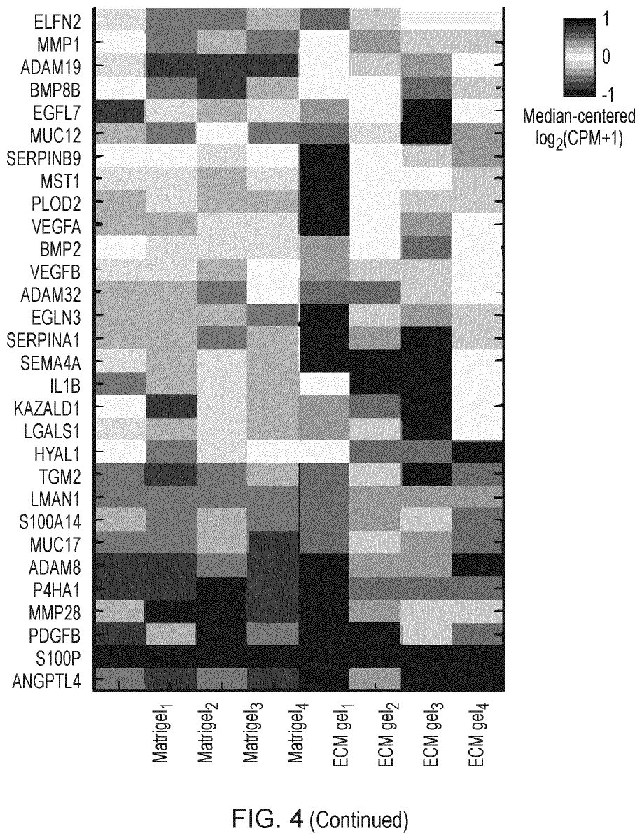

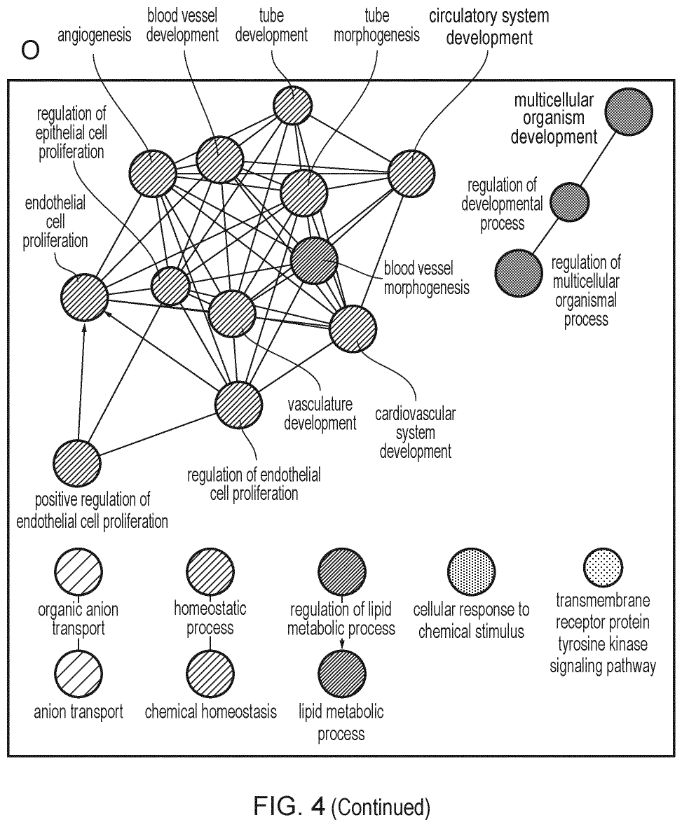

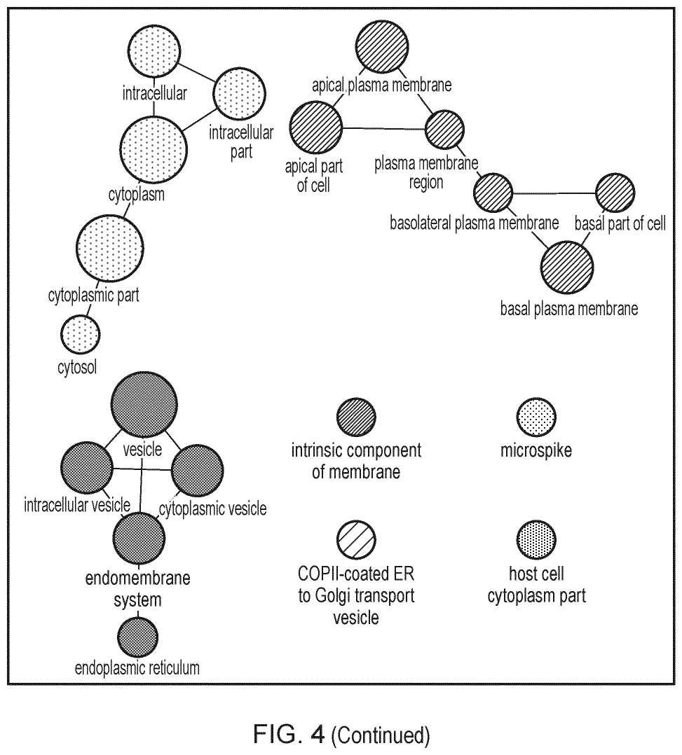

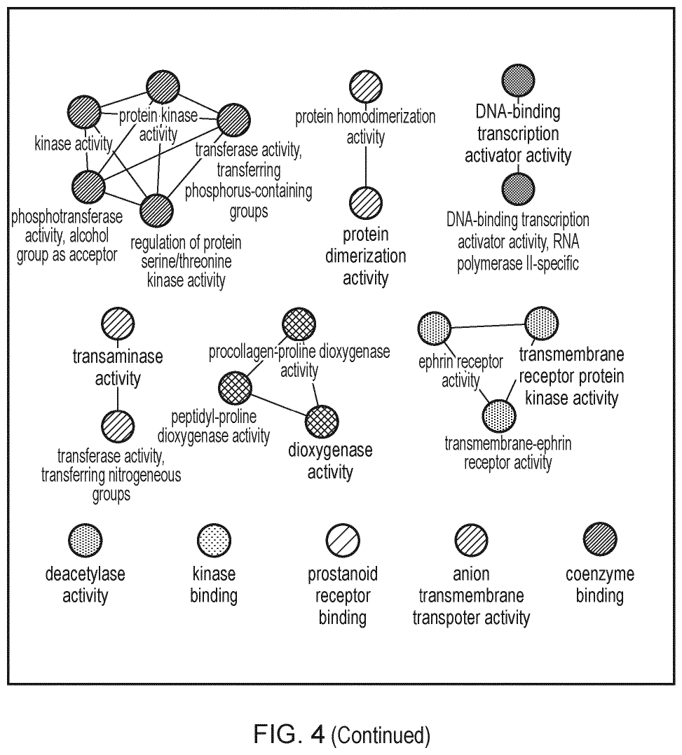

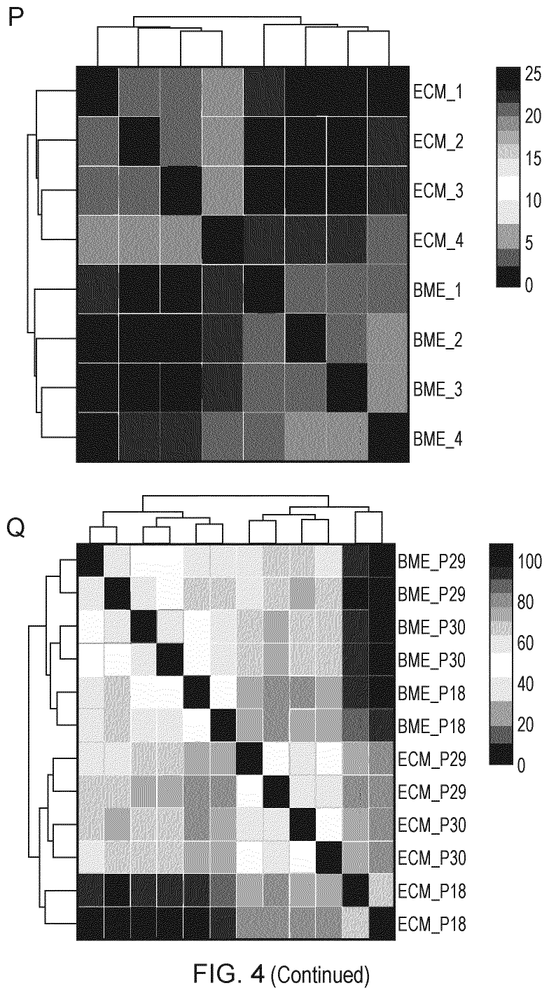

To better understand the behavior of human organoids in ECM gel compared to Matrigel, the cultures were further characterized through transcriptomic and functional analyses. 3′ RNA-sequencing was performed on human small intestinal organoids derived from a pediatric donor (FIG. 4A-E). PCA showed that, despite sample-to-sample variability, the two groups of samples were clearly separated according to the first principal component (FIG. 4A). 1833 genes were found differentially expressed, but only 388 had an absolute fold change greater than 2, of these 173 and 215 were up- and down-regulated in ECM conditions, respectively (FIG. 4B). Few gene sets related to processes that could be relevant for cell adaptation and differentiation within organoids²⁶ were selected. As shown in FIG. 4C, multiple of these genes were found differentially expressed. Interestingly, while LGR5 was comparable in both conditions, other crypt markers such as OLFM4, SMOC2 and LYZwere statistically overexpressed in ECM gel cultured organoids. Transit amplifying region markers (BMI1, LRIG1) were comparable with Matrigel controls. Differentiated intestinal cell markers were partly comparable between the two conditions (FABP1, MUC1, MUC3A, MUC5B) or slightly overexpressed in Matrigel compared to ECM gel cultured organoids (EZR, VIL1, MUC12, MUC13, MUC17, MUC20). Of the differentiation markers, only CHGA resulted overexpressed in ECM gel.

Of all preserved proteins identified in the decellularized ECM, 109 (˜6% of all DEGs) were also identified as differentially expressed at the transcriptomic level, with 42 up-regulated in ECM and 67 up-regulated in Matrigel. As for the transcripts known to be in the core matrisome²⁷ (FIG. 4D), the only 4 transcripts (TINAGL1, LTBP4, CRELD1, ECM1) that were both DEGs and identified as proteins, were all up-regulated in organoids cultured in Matrigel. Only TINAGL1 (IPI00115458) was identified in Matrigel in a previous proteomic study²⁸. Moreover, 2 (LTBP4, ECM1) of these 4 transcripts were highlighted in FIG. 2C as characterizing cluster 3, the one mainly incorporating endoderm-derived tissues. Only 5 transcripts (LGALS1, LGALS3, LMAN1, P4HA1, TGM2), out of the 109, belong to the matrisome-associated gene set, and were also all up-regulated in Matrigel organoids at the transcriptomic level, despite 4 of these proteins have been previously identified also in Matrigel (Lgals1, IPI00229517; Lgals3, IPI00131259; Lman1, IPI00132475; P4ha1, IPI00272381). Matrisome-associated (e.g. Remodeling enzymes) differentially expressed genes complete panel is shown in FIG. 4N. An unbiased analysis of GO categories over-represented in the identified DEGs at transcriptomic level highlighted multiple functional categories related to processes occurring at the cell-extracellular environment interface (FIG. 4E, FIG. 4O). Interestingly, some of the identified processes could be relevant in the ECM gel role of organoid support, like those involved in “Vasculature development” or “Multicellular organism development”.

These outcomes on human SI organoids outline the strict connection between microenvironment and cell physiology, therefore moving to a native ECM might benefit organoid phenotype. It was also checked by real-time PCR the differential expression of some major intestinal markers observed in the SI RNA-sequencing in FIG. 4c and FIG. 4f. In this analysis, LGR5 and CHGA resulted overexpressed in ECM gel, while LYZ was comparable. ALPI and MUC2 were both overexpressed in Matrigel. These data confirm the previous observation of a higher fraction of crypt/stem cells present in ECM-cultured human SI organoids.

Moreover, a full set of transcriptomic data on human liver cells is reported. For this, human adult cholangiocyte ducts, and human fetal hepatocyte organoids, previously presented in FIG. 3C, were analysed. Bulk 3′ RNA-sequencing was performed with comparison of the 2 liver cell types cultured in ECM vs BME. Regarding the RNA-seq analysis for the human ductal organoids, while the PCA plot and the heatmap of the differentially expressed genes (FIG. 4G-H) showed that the organoids cultured in ECM gel were slightly different from those cultured in BME (based on PC1), none of the critical ductal markers (KRT7, KRT8, KRT18, KRT19, EPCAM) were downregulated (FIG. 4I). The cluster map of human ductal liver organoids cultured in ECM gel vs BME is shown in FIG. 4P. SOX9 and TACSTD2 (TROP2) were significantly upregulated in the ECM gel culture condition (FIG. 4I). Both are markers of progenitor-like cells, where TROP2 has been recently described as a marker of bipotent progenitors²⁹. The cluster map of human ductal liver organoids cultured in ECM gel vs. BME is shown in FIG. 4(P). Upregulation of SOX9 and TACSTD2 may be advantageous, to allow for more “multi-potent” population in expansion.

The RNA-seq analysis for the human fetal hepatic organoids highlighted also in this case a distance between ECM gel and BME cultured organoids, as shown in the PCA plot and in the heatmap of the differentially expressed genes (FIG. 4J-K). In this analysis two separate fetal lines, KK2 and KK3, were compared, and the observed distance might also be ascribed to donor-related differences. Nonetheless, none of the specific hepatocyte markers²⁴ (ALB, ASGR1, ASGR2, SERPINA1, FABP1, APOA2) showed any differential expression (FIG. 4L). The cluster map of human fetal hepatic organoids cultured in ECM gel vs BME is reported in FIG. 4Q.

Interestingly, the functional analysis on the production of human albumin by both ductal²³ and hepatic²⁴ organoids showed a comparable secretion in both ECM gel and BME cultures (FIG. 4M). Real Time PCR was used to check the differential expression of some major intestinal markers observed in the small intestinal RNA-sequencing in FIG. 4C and FIG. 4F). In this analysis, LGR5 and CHGA resulted overexpressed in ECM gel, while LYZ was comparable. ALPI and MUC2 were both overexpressed in Matrigel. These data confirm the previous observation of a higher fraction of crypt/stem cells present in ECM-cultured human small intestinal organoids.

### Example 5—In Vivo Delivery of ECM Cultured Organoids

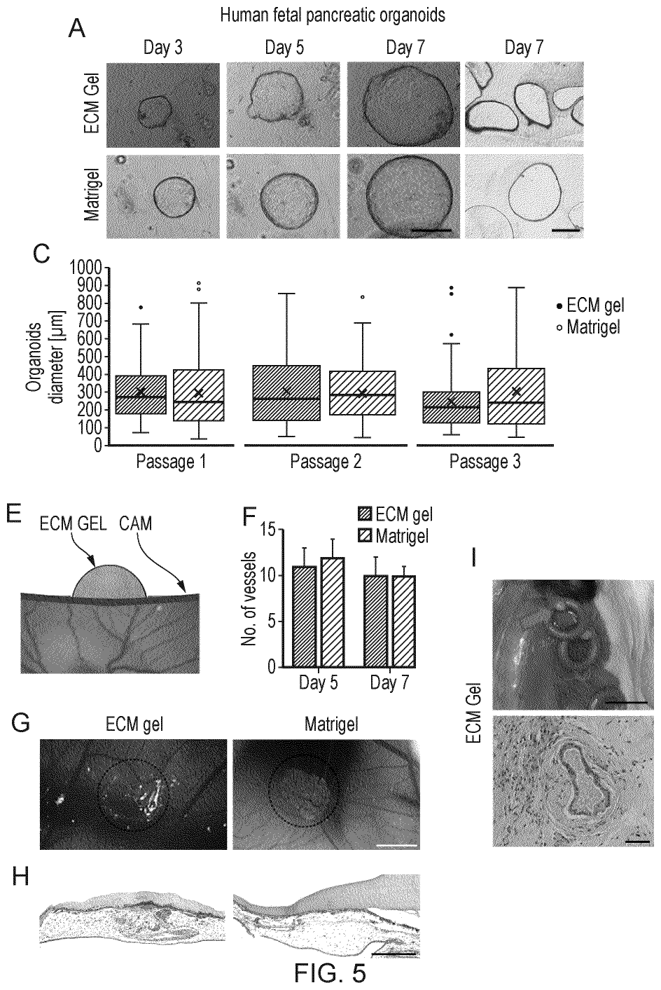

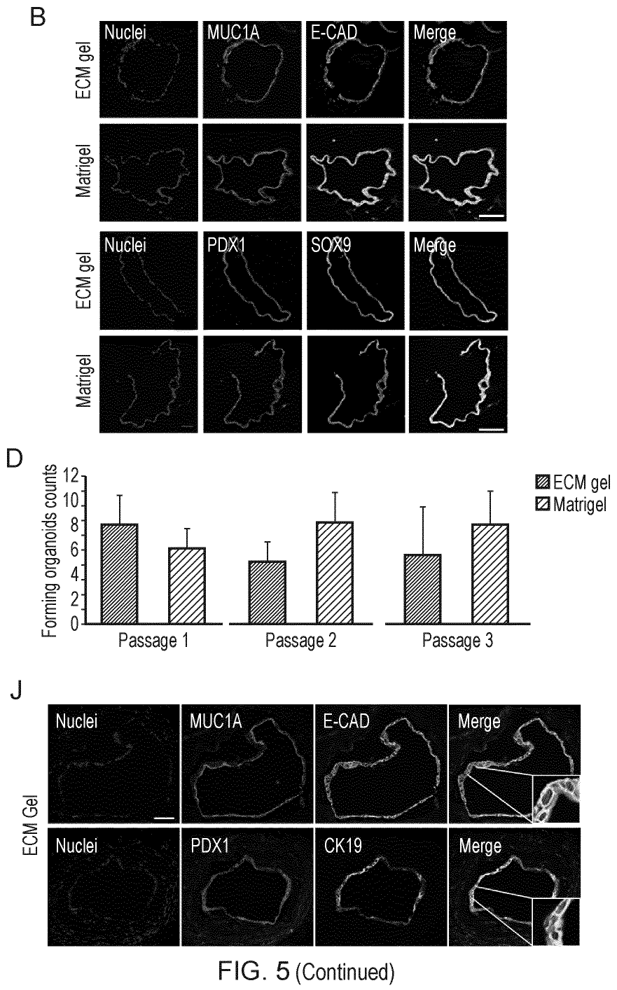

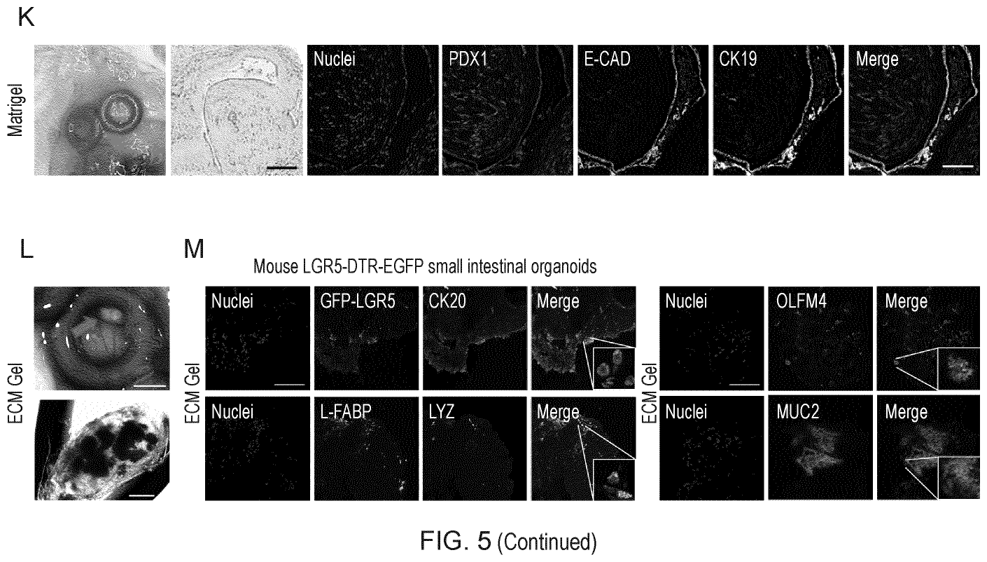

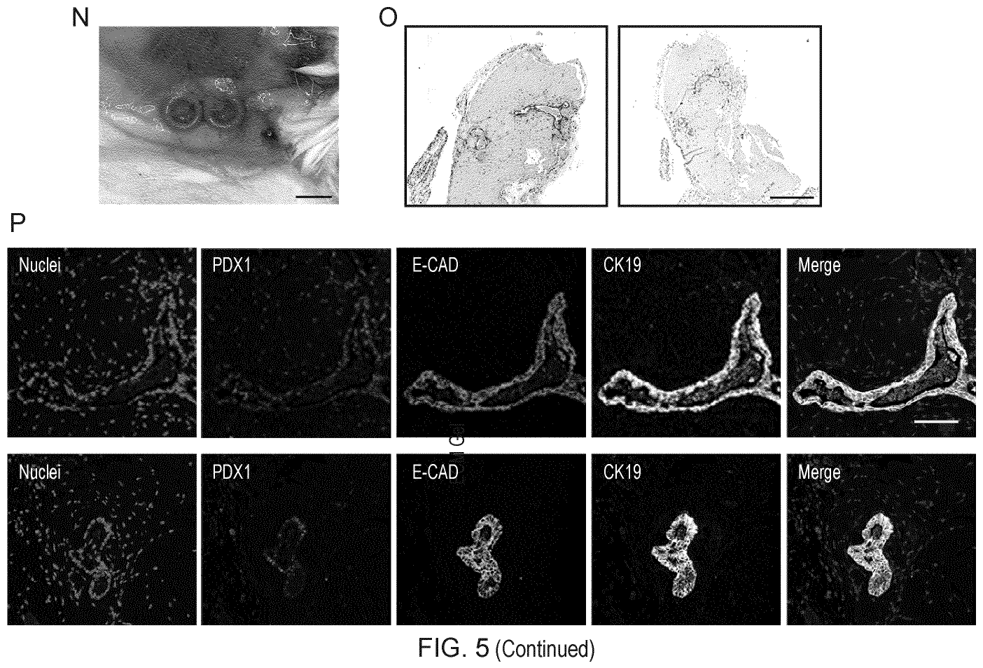

In vivo delivery of cultured human organoids was explored, which remains challenging as it is linked to efficient vascular support, and the possibility of using clinically compatible vectors. For this purpose, human fetal pancreatic organoids were used as a test system. Fetal ducts grown in vitro in ECM gel and Matrigel showed similar morphology (FIG. 5A). ECM allowed maintenance of similar expression of lineage-specific markers such as mucin-1a, epithelial cadherin, together with pancreatic-duodenal homeobox 1 (PDX1) and SOX9 (FIG. 5B). Cells were successfully expanded for at least 3 passages during which they maintained comparable organoids size (FIG. 5C) and numbers (FIG. 5CD) in ECM gel and control.

In order to evaluate the angiogenic potential of ECM gels, the Chick Chorioallantoic Membrane (CAM) assay was performed (FIG. 5E). When compared to Matrigel over 5 and 7 days, ECM gel showed no difference in the number of new vessels formed (FIG. 5F). Morphological (FIG. 5G) and histological (FIG. 5H) characterization of the gel and control on the CAM showed no difference.

To further evaluate the in vivo potential, pancreatic organoids within ECM gels (and Matrigel as control) were also seeded, and transplanted subcutaneously in immunodeficient mice. Cells were then harvested at 2.5 (FIG. 5I) and 8 weeks (FIG. 5N-0). In both time points, the preservation of organoid organization and comparable expression of epithelial (e-cadherin), pancreatic (mucin-1A and cytokeratin-19) markers was observed, along with transcription factors (insulin promoter factor 1) between ECM gel and Matrigel (FIG. 5J-K,FIG. 5P).

As an additional application of this model, a set of in vivo experiments with small intestinal organoids was performed. To this end, the LGR5-DTR-EGFP mouse model³⁰ was used. Mouse small intestinal organoids with GFP-reporter crypt stem cells, which could be traced after an in vivo transplant, were derived. These cells were transplanted in ECM gels, into mice back sub-cutaneous pockets. After one month, all 5 ECM gels transplanted were retrieved, which contained matured organoids (FIG. 5L). Retrieved cells showed an active stem compartment highlighted by the presence of anti-GFP for LGR5+ cells, double checked with olfactomedin-4. Paneth cells were present (marked with lysozyme), and we highlighted also the presence of differentiated cell types such as enterocytes and goblet cells, marked with L-type fatty acid binding protein (L-FABP), cytokeratin-20 and mucin-2 (FIG. 5M).

### Example 6—Discussion and Materials and Methods

Discussion

Disclosed here is the successful development of ECM gels that have the potential to both direct and influence human organoids behavior in vitro and in vivo. This includes directing cell adhesion, survival, proliferation, and differentiation, while also providing a mechanical support to the cells. An ex vivo 3D cell culture support should ideally recapitulate aspects of this native microenvironment and facilitate these functions³¹. LGR5+ cells, isolated from the crypts of the intestine are an example of a cell type that favors a 3D environment for ex vivo culture over 2-D³². A 2D culture, provides an unnatural environment for the cells. In a monolayer culture, only a portion of the cell surface is in contact with ECM and neighboring cells, with the remaining portion exposed to the culture media. This provides a homogeneous supply of nutrients, cytokines and growth factors to this external membrane, which unlikely resemble the dynamic spatial gradient of nutrient supply received in vivo³³.

Both natural and synthetic hydrogels have been examined for their ability to support organoid culture, each having their own associated advantages and limitations. Recently, synthetic alternatives to Matrigel have been reported^{9,10}. While synthetic gels have the advantage of being GMP-compliant and reproducible, they are limited by a lack of biological signals provided to the cells. ECM is far more complex and this disclosure demonstrates that this information allowed clustering within the germ layer of derivation. The ECM gels of the disclosure can support the culture not only of intestinal organoids, but also cells derived from other endoderm-derived tissue such as liver, stomach and pancreas.

Porcine intestine tissue was decellularized using the DET protocol as disclosed herein. Mesentery and the external muscle layer were removed in situ using an in-house established protocol. One cycle of the DET protocol was required to remove nuclei from the scaffold which was confirmed with H&E staining along with a significant reduction in DNA content. Histological analysis confirmed the presence of collagen, elastin and GAGs post gelation. Collagen increase compared to tissue weight is a common feature following decellularization and loss in cytoplasmic compartment. Further characterization highlighted maintenance of the main collagen isoforms⁸. Spectrophotometry and rheology experiments confirmed gelation of the ECM hydrogel at all concentrations. Gelation also preserved appropriate stiffness which is fundamental for enteroid formation, survival and differentiation.

Past works describe how ECM is not only a mere scaffold, but it is an integral determinant of tissue specificity itself. Epithelial and mesenchymal components interact during development to direct tissue morphogenesis and differentiation. The tissue development is not a cell autonomous process, but it is instead instructed by the surrounding environment³⁴. Extensive proteomic analysis on the decellularized tissue powder further confirmed that the major extracellular matrix components were preserved, such as the main collagen isoforms, but also non-extracellular matrix components, which may play a role in the signal transduction of the organoids cultured in the gel. Small intestinal ECM proteomic profile clusters with the main endoderm-derived organ's ECM profiles. This characteristic suggests that this gel could also be used to support the culture of both mouse and most relevantly human organoids derived from stomach, adult (ducts) and fetal (hepatocytes) liver, adult and fetal small intestinal mucosa, and fetal pancreas. Many exosomal proteins were also preserved within the decellularized matrix, including proteins related to cell adhesion. The metabolomics analysis on the digested powder allowed sub-products of protein degradation and fatty acid residual to be identified, which were expected to be found after cell membrane breakdown during decellularization process.

Transcriptomic analyses performed on ECM gel cultures showed how human organoids maintain their identities compared to Matrigel cultures, and this is confirmed in human small intestine, liver duct, and hepatic organoids. Interestingly, pediatric small intestinal organoids maintained a higher proportion of crypt stem cells in SI ECM hydrogels compared to slightly more differentiated cells in Matrigel. This may advantageously allow for more efficient propagation of a culture. Nonetheless, all the specific intestinal markers, defining both crypt and villi signature, were expressed in ECM gel compared to Matrigel cultured organoids, underlying the suitability of the ECM to host human cultures.

On the other hand, ECM specific markers that were detected at the proteomic level in the decellularized ECM were found to be all overexpressed in Matrigel cultured SI organoids. Only few of these were previously reported to be present in Matrigel²⁸. This observation might be ascribed to the necessity of SI organoids to produce their intestinal extracellular matrix, compared to ECM gel cultured organoids that are already integrating signals from the native small intestinal ECM.

A translational application of ECM-derived hydrogels is currently hampered by the high variability of the lab-derived products. In our study, to better standardize the process we always used similar age and similar weight (3 kg) piglets of the same pure ‘Petrain’ breed. Each new batch was then tested for ECM digestion quality, gelation quality, stability in culture medium in incubator and cytocompatibility with organoids.

We furthermore combined the ECM hydrogel of the invention with synthetic molecules, for example photo-polymerizable polyacrylamide to obtain a combined gel suitable for monolayer growth of mouse and human small intestinal organoids. Based on these results it appears other synthetic materials could be used e.g. polyglycolic acid (PGA), polylactic acid (PLA).

Long term expansion requires stable and consistent cultures. In some embodiments, the cultures displayed comparable outcome during the first 3-4 passages, which would be sufficient for ex vivo cell expansion. Moreover, this did not affect in vivo delivery of the cells, which is ultimately one of the main objectives of the ECM gel.

In view of GMP-grade production for clinical use, it is important to underline that all the chemicals and reagents utilized during each step of the gel production pipeline are already commercially available at GMP-grade. This list is presented in the following table.

| Good Manufacturing Practice (GMP) | | Product | Pack | |
|---|---|---|---|---|
| products | Company | code | size | |
| Milli-Q ® Integral ultrapure water Type I, | Merck Millipore | ZRXQ015WW | unlimited | |
| 0.22 μm filtered | | | | |
| Sodium deoxycholate (SDC) PharmaGrade | Merck - Sigma | S1827 | 1 | kg |
| manufactured under appropriate controls | Aldrich | | | |
| Sodium chloride (NaCl) PharmaGrade, | Merck - Sigma | RES0926S- | 25 | kg |
| USP, Manufactured under appropriate GMP | Aldrich | A705X | | |
| controls | | | | |
| DNase I, recombinant, RNase-free, GMP | Roche - | 03724751103 | 4 | kU |
| Grade | CustomBiotech | | | |
| GMP-certified and virus validated Pepsin, | Biofac | Pepsin | 1 | kg |
| pharmaceutical and technical grade | | Powder - | | |
| | | GMP grade | | |
| Probumin ® Bovine Serum Albumin (BSA) | Merck - Sigma | 820476 | 5 | kg |
| Biotech Grade | Aldrich | | | |
| Hydrochloric acid (HCI) Grade ACS | Merck - Sigma | 30721-1L-M | 1 | L |
| reagent, reag. ISO, reag. Ph. Eur. 37% | Aldrich | | | |

Importantly, as a further step towards the applicability to a clinical translatable protocol, this disclosure demonstrates the possibility to derive organoids from human biopsies without the use of Matrigel at the first passage after tissue dissociation.

Further, human organoids cultures of the disclosure can survive in vivo maintaining both structure and signature expression at protein level. Importantly, angiogenesis occurred already at 2.5 weeks post-transplantation in vivo and increased over time with no major differences between ECM gel and Matrigel.

The in vivo results disclosed highlight the utility of the ECM gel of the disclosure to efficiently deliver cells of both human and animal origin. Moreover, the ECM gel of the disclosure facilitated cell survival of up to 2 months for enteroids derived from different organs, as shown with the fetal pancreatic ductal organoids, and the small intestinal organoids.

Materials and Methods

Porcine Intestinal Tissue Collection

Porcine (Sus scrofa domesticus) small intestinal (SI) mucosal/submucosal layers from the ‘Pietrain’ breed were used. Piglets up to 3 kg in weight were euthanized via blunt trauma once the criteria outlined by the JSR veterinary advisors had been met. Once sacrificed, the animals were transported to the lab via courier and the intestine was harvested immediately on arrival (within 6 hours of euthanasia). The whole small intestine was harvested (duodenum, jejunum, and ileum) and the internal tube was pulled out leaving behind the external layer and mesentery. The retrieved mucosal/submucosal tissue was then extensively cleaned with pressurized water, opened longitudinally, cut into 5 cm or 4 cm or 3 cm or 2 cm or 1 cm pieces and placed in Milli-Q® (Merck Millipore) water overnight at 4° C., on a laboratory rotator, or in a magnetic stirrer or agitator to begin the first step of decellularization. Each batch of ECM was composed of 3 pooled piglets SI.

Decellularization of Porcine Intestine Tissue

The detergent-enzymatic treatment (DET) for decellularization, previously established on rat small bowel was optimized for the porcine intestine²⁰. After the first overnight wash, the tissue was decellularized at 4% sodium deoxycholate (Sigma Aldrich) for 4 hr at room temperature (RT). This was followed by a washing step in Milli-Q water for 24 h at RT, with multiple water changes throughout, and then a step of 2000 kU DNase-I (Sigma Aldrich) in 1M NaCl (Sigma Aldrich) for 3 hr at RT. The tissue was then placed in Milli-Q water and washed for 2 days or 3 days, with multiple water changes. Wash steps are important for removing any cytotoxic residual of sodium deoxycholate. A laboratory rotator or magnetic stirrer was used throughout the decellularization process.

Gelation Protocol and Cell Inclusion

The decellularized porcine intestine was freeze dried for 72h (Labconco FreeZone Triad Freeze Dry Systems), milled into a thin powder using a mini mill (Thomas Wiley, mesh 40), sterilized by gamma irradiation (17 kGy for 10h) and stored at −20° C. until further use. For gelation, the ECM powder was digested at 4 or 6 or 8 or 10 mg/ml in pepsin/HCl solution (1 mg/ml in 0.1M HCl) at RT for 72 hours, in constant rotation. For preparing organoid cultures, the ECM powder was digested at a concentration of less than 8 mg/mL. Pre-gel was then centrifuged (200-400 g for 3-5 min) to precipitate and discard eventual undigested particles. Acidic pre-gel solution was commonly used freshly prepared, but it could be stored at 4° C. up to 1 month, or frozen in aliquots at −20° C. for prolonged storage. For cell seeding, while working on ice and immediately prior to use, pre-gel solution was equilibrated to cytocompatible salinity adding 10% 10X PBS for mechanical tests, or 10X DMEM F/12 (Thermo Fisher) for cell culture and neutralized to physiological pH of 7.5 by addition of NaOH 10M and thoroughly mixing, with modification of published protocols^{18,19}. During these steps, cut-end pipette tips are used, to facilitate dense gel pipetting. Gel was mixed with cell pellets and aliquot in 30-40 μL droplets in Petri dish. Gelation took place in 30 min in the incubator. Organoids were optionally cultured in 4-6 mg/mL ECM gels.

Tissue Histology

Tissue samples were taken at random immediately post-harvesting and after each cycle of decellularization. For paraffin embedded sections, samples were fixed in 4% paraformaldehyde solution in PBS for 24 hours at RT, washed in dH₂O, dehydrated in graded alcohol, embedded and cut into 5 μm sections. For frozen sections, samples were snap frozen in liquid nitrogen, placed in OCT and cut into 7 μm sections. For ECM gel and Matrigel® Basement Membrane Matrix Growth Factor Reduced (GFR) (Corning 354230), droplets were fixed in glutaraldehyde 2% for 2 h. After fixing, another PBS wash was followed with 100-150 μL of 2% agarose solution until the droplet was fully covered. The agarose was removed, taking with it the gel droplet and stored in 70% ethanol. The agarose/hydrogel samples were then dehydrated with a series of ethanol washes with increasing concentrations followed by two xylene washes. Samples were embedded in paraffin and cut into 7 μm sections. Tissue slides were stained according to manufacturers' instructions with Hematoxylin and Eosin (H&E) (Thermo Fisher) and Hoechst 33342 (Thermo Fisher) to determine the presence of nuclei and Picrosirius Red (PR), Elastic Van Gieson (EVG) and Alcian Blue (AB) (Thermo Fisher) to assess retention of collagen, elastin and glycosaminoglycans respectively.

DNA and ECM Quantification

Tissue samples were taken at random immediately post-harvesting and after decellularization protocol for DNA and ECM components quantification. DNA was quantified using a PureLink Genomic DNA Mini Kit (Thermo Fisher). The final concentration of DNA in the samples was measured using a NanoDrop (model NanoDrop 1000 Spectrophotometer by Thermo Fisher). ECM components were quantified using a QuickZyme Collagen assay kit (QuickZyme Biosciences) to measure the collagen, a Blyscan Sulfated Glycosaminoglycan Assay kit (Biocolor) for the glycosaminoglycans (GAGs) and a Fastin Elastin Assay kit (Biocolor) for elastin, according to manufacturers' instructions.

Turbidity

The turbidity of the hydrogels was assessed using spectrophotometry (n≥5). 200 μl of the hydrogel were pipetted into a 96-well plate and absorbance at 450 nm was measured at 37° C. once per min for 1 hr³⁶. Readings were normalized to a PBS control and then normalized using the calculation below, where NA is the final normalized absorbance, R is the absorbance reading obtained at a given time, Rₘᵢₙ is the smallest absorbance value recorded and Rₘₐₓ is the greatest absorbance value. From the data, the half gelation time (t1/2), the gelation rate (S) and the lag time (tlag) was calculated.

$$NA = \frac{R - {R}_{m i n}}{{R}_{m a x} - {R}_{m i n}}$$

Oscillatory Rheology

The neutralized gel (3 mL) was placed in between the two plates of the rheometer heated to 37° C. with a gap size of 1 mm, and a sinusoidal stress of constant maximum amplitude of 0.5 Pa applied at a frequency of 1 Hz. The resulting strain was measured for approximately 1 hour 30 minutes³⁶.

Oscillatory rheology was performed as a temperature ramping study using a Discovery HR-2 rheometer (TA instruments). 1 mL of the neutralized digested hydrogel was poured onto the preheated steel peltier plate, 4° C. for Matrigel (100% concentration) and for the ECM gel. The 40 mm parallel plate is lowered to a gap of 650-800 μm, or until the gel perfectly fills the gap. The sinusoidal stress of constant 21 maximum amplitude of 50 Pa was applied at frequency of 25 Hz, with a temperature ramp from 22-37° C. for 7.5 minutes, a constant temperature of 37° C. for 45-75 min and finally another temperature ramp from 37-50° C. for a period of 7.5 min. G′ and G″ were measured for the entire period.

Scanning Electron Microscopy (SEM)

SEM-images of the cross-section, top and bottom surfaces of the ECM gel were taken to examine surface-topography of the material. All samples were fixed in 2.5% glutaraldehyde (Sigma Aldrich) washed in 0.1 M phosphate buffer (pH 7.4), post fixed with 1% OSO₄ (osmium tetraoxide)/1.5% Potassium Ferrocyanide K₄[Fe(CN)₆] in 0.1 M phosphate buffer followed by a dH₂O wash. Specimens were then dehydrated in a graded ethanol-water series to 100% ethanol (50%, 60%, 70%, 80%, 90%, 95%, 100%) and critical point-dried using CO₂³⁷. The samples were mounted onto aluminum stubs using sticky carbon tabs, oriented so the surfaces of interest were presented to the beam. Samples were coated with a 2 nm-thin layer of Au/Pd using a Gatan ion-beam coater, and viewed using a Jeol 7401 FEG-SEM.

Chick Chorioallantoic Membrane (CAM) Assay

Fertilized chicken eggs of ‘White Leghorn’ breed (Henry Stewart and Co.) were incubated in a MultiQuip Incubator (E2) at 37° C. with 60% constant humidity³⁸. Ethics approval was obtained by the University College London Animal Ethics Committee. A small window was made in the shell on day 3 of chick embryo development under aseptic conditions. The window was resealed with adhesive tape and eggs were returned to the incubator until day 8 of chick embryo development. On day 8, ECM gels and Matrigel grafts were placed on top of the CAM and eggs were resealed and returned to the incubator. On day 10 PBS was added to the CAM to avoid the CAM drying out. Pictures were taken on day 13 and day 15. On day 15 ECM gel and Matrigel grafts with surrounding CAM were harvested from each embryo and fixed with 4% paraformaldehyde before paraffin embedding. Serial 5 μm sections were stained with H&E. Slides were digitally scanned using the NanoZoomer (Hamamatsu Photonics K.K.).

Proteomic Sample Pre-Processing

The lyophilized ECM powder was split into 3 biological replicates, each processed independently and analyzed in triplicate by LC-MS/MS. The powder was resuspended in lysis buffer and two spike-in proteins (each at 0.5 μg/100 μg of protein powder) were added: carnitine monooxygenase oxygenase subunit (cntA, D0C9N6) from Acinetobacter baumannii, and CTP synthase (CTPsyn, Q9VUL1) from Drosophila melanogaster. Protein extraction was performed by heating at 90° C. for 10 min, and centrifuging at maximum velocity for 10 min at 4° C. ECM-derived proteins were reduced in 0.1 M dithiothreitol (DTT) at 95° C. for 5 min, dissolved in 8 M urea solution after cooling down to room temperature, alkylated with 55 mM iodoacetamide for 30 min at 25° C. in the dark. Alkylated proteins were purified using Microcon YM-10 filter unit (MRCPRT010, Millipore) for 8 times at 14000 g for 40 min³⁹ followed by trypsin (Promega) digestion for 16 h at 37° C. pH was adjusted to 3 by addition of formic acid. Peptides were desalted by C-18 column and dried into powder and were then re-suspended in 30 μl 0.1% acetic acid for the following mass spectrometry analysis.

Proteomic LC-MS/MS Analysis

Protein identification by liquid chromatography-tandem mass spectrometry (LC-MS/MS) was performed using Thermo Fusion Mass Spectrometer with Thermo Easy-nLC1000 Liquid Chromatography. 130 min of LC-MS gradients were performed by increasing organic proportion. The first level of MS was detected by Orbitrap with parameter of Resolution at 120K, Scan Rang at 300-1800 m/z, Mass Tolerance at 10 ppm. The second level of MS was isolated by Quadrupole, activated by HCD and detected by Orbitrap. The Orbitrap Resolution for the second level of MS was 30K.

Proteomic Bioinformatics Analysis

The mass spectrometry-derived data were searched against a human protein database (Uniprot Homo sapiens reference proteome, UP000005640) by MaxQuant v. 1.6.7.0⁴⁰. Oxidation of methionine residues and acetyl of protein N-term were set as variable modifications. Carbamidomethyl on cysteine was set as fixed modification. Peptide-spectrum matches (PSMs) were adjusted to a 1% and then assembled further to a final protein-level false discovery rate (FDR) of 1%. Intensity-based absolute quantification (iBAQ)⁴¹ was normalized according to the mean quantification of the two spiked-in proteins. Proteins with less than 2 unique peptides identified were filtered out. The remaining 1617 proteins were used in the subsequent analysis. Mean and standard error of the mean among replicates were calculated in MATLAB R2017a based on normalized iBAQ intensity values, then recalculated in percentage. Overrepresentation analyses of all and exosomal proteins was performed in DAVID 6.8⁴². Proteins from Gene Ontology-Cellular Component (GO-CC) categories ECM (GO-CC:0031012) and extracellular exosomes (GO-CC:0070062) were selected. A hierarchical clustering analysis of the expression of this ECM protein set in native tissues from a recent draft map of the human proteome⁴³ (online resource available at http://www.proteomicsDB.org) was obtained using the web-based tool “Expression heatmap”⁴⁴, reporting protein expressions in different tissues quantified as iBAQ at the protein level. A principal component analysis (PCA) was performed in MATLAB using log 10 iBAQ intensity values from our data and from http://www.proteomicsDB.org, both within GO-CC:0031012.

Metabolomics

30 μL ECM powder pepsin-digested (pre-gel) were analyzed by gas chromatography—mass spectrometry (GC-MS) based metabolomics using a standard protocol⁴⁵ on a Thermo Trace GC and DSQ II mass spectrometer. Chromatographic deconvolution, alignment and database matching was performed using MS-DIAL 3.90⁴⁶.

Culture of Mouse Intestinal Organoids

CD1 mice and LGR5-DTR-EGFP mice³⁰ were sacrificed by cervical dislocation and the intestine was harvested from the pylorus to the caecum. The obtained tissue was washed through once with ice-cold PBS, cleared of any mesenteric or fatty tissue and cut longitudinally. Following a further series of PBS washes, a cover slip was used to shave away the villi and the remaining tissue was cut into 2-3 mm pieces and washed vigorously. This was then incubated in 2 mM ethylenediaminetetraacetic acid (EDTA) in PBS for 30 minutes followed by vigorous shaking for 5 min in PBS. The obtained supernatant, containing the intestinal crypts, was centrifuged at 800 rpm for 5 minutes at 4° C. (Hettich zentrifugen Rotina 420). The pellet was washed once with basal media (Advanced DMEM/F12 media, supplemented with 1% of each GlutaMAX, HEPES and Penicillin/Streptomycin) and centrifuged at 1000 rpm. The pellet was re-suspended in Matrigel growth factor reduced and plated onto a 24-well plate. Primocin 1X (Thermo Fisher) and ROCK inhibitor 10 μm are added after isolation. For medium recipe look in table 2.

TABLE 2
Mouse small intestinal organoid medium

| Component | Stock conc. | | Final conc. | |
|---|---|---|---|---|
| Advanced DMEM F-12 (Thermo 12634) | — | | To volume | |
| HEPES (Thermo 15630080) | 1M | | 10 | mM |
| Glutamax (Thermo 35050061) | 100 | X | 2 | mM |
| B-27 supplement minus vitamin A | 50 | X | 1 | X |
| (Thermo 12587010) | | | | |
| n-acetylcysteine (Sigma A9165) | 500 | mM | 1.25 | mM |
| Pen/Strep (Thermo 15140122) | 100% | | 1% | |
| Wnt-3A (Peprotech 315-20) optional | 50 | μg/mL | 100 | ng/mL |
| R-spondin 1 (Peprotech 120-38) | 100 | μg/mL | 500 | ng/mL |
| Noggin (R&D 6057-NG) | 100 | μg/mL | 100 | ng/mL |
| EGF (Thermo PMG8043) | 500 | μg/mL | 50 | ng/mL |

Culture of Pediatric Human Intestinal and Human Gastric Organoids

Human Pediatric Samples from Small Intestine and Stomach were Collected after Consent following the guidelines of the license 08ND13 and 18DS02. Small intestinal crypt stem cells and gastric crypt stem cell were isolated from pediatric biopsies following well-established dissociation protocols^{47,48}. Isolated crypts at first passage (p0) were cultured in Matrigel growth factor reduced droplets, or in 4 mg/mL ECM gel. For media recipes look in table 3 and 4.

TABLE 3
Human pediatric and fetal small intestinal organoid medium

| Component | Stock conc. | | Final conc. | |
|---|---|---|---|---|
| Advanced DMEM F-12 (Thermo 12634) | — | | To volume | |
| HEPES (Thermo 15630080) | 1M | | 10 | mM |
| Glutamax (Thermo 35050061) | 100 | X | 2 | mM |
| B-27 supplement minus vitamin A | 50 | X | 1 | X |
| (Thermo 12587010) | | | | |
| n-acetylcysteine (Sigma A9165) | 500 | mM | 1.25 | mM |
| Pen/Strep (Thermo 15140122) | 100% | | 1% | |
| Wnt-3A (Peprotech 315-20) | 50 | μg/mL | 100 | ng/mL |
| R-spondin 1 (Peprotech 120-38) | 100 | μg/mL | 500 | ng/mL |
| Noggin (R&D 6057-NG) | 100 | μg/mL | 100 | ng/mL |
| EGF (Thermo PMG8043) | 500 | μg/mL | 50 | ng/mL |
| Gastrin (Sigma G9020) | 100 | μM | 10 | nM |
| GSK-3 inhibitor (CHIR 99021) | 3 | mM | 3 | μM |
| (Tocris 4423) | | | | |
| TGFb inhibitor (A83-01) | 500 | μM | 500 | nM |
| (Sigma SML0788) | | | | |
| P38 inhibitor (SB202190) (Sigma S7067) | 30 | mM | 10 | μM |
| Prostaglandin E2 (Cambridge cay14010) | 100 | μM | 10 | nM |

TABLE 4
Human pediatric gastric organoid medium

| Component | Stock conc. | | Final conc. | |
|---|---|---|---|---|
| Advanced DMEM F-12 (Thermo 12634) | — | | To volume | |
| HEPES (Thermo 15630080) | 1M | | 10 | mM |
| Glutamax (Thermo 35050061) | 100 | X | 2 | mM |
| B-27 supplement minus vitamin A | 50 | X | 1 | X |
| (Thermo 12587010) | | | | |
| n-acetylcysteine (Sigma A9165) | 500 | mM | 1.25 | mM |
| Pen/Strep (Thermo 15140122) | 100% | | 1% | |
| Wnt-3A (Peprotech 315-20) | 50 | μg/mL | 100 | ng/mL |
| R-spondin 1 (Peprotech 120-38) | 100 | μg/mL | 500 | ng/mL |
| Noggin (R&D 6057-NG) | 100 | μg/mL | 100 | ng/mL |
| EGF (Thermo PMG8043) | 500 | μg/mL | 50 | ng/mL |
| Gastrin (Sigma G9020) | 100 | μM | 10 | nM |
| GSK-3 inhibitor (CHIR 99021) | 3 | mM | 3 | μM |
| (Tocris 4423) | | | | |
| TGFb inhibitor (A83-01) | 500 | μM | 5 | μM |
| (Sigma SML0788) | | | | |
| FGF10 (Peprotech 100-26) | 100 | μg/mL | 200 | ng/mL |

Culture of Human Hepatic Organoids and Cholangiocyte Organoids

Liver organoids were cultured following the protocol previously published^{23,24}. Hepatic organoids were split by gentle dissociation with TrypLE Express (Thermo Fisher), while ductal organoids are passaged by manual disruption. Organoids were seeded in ECM gels, Matrigel and Cultrex® 3-D Culture Matrix™ basement membrane extract (BME), both at 100% concentration, as controls. For media recipes look in table 5 and 6.

TABLE 5
Human ductal organoid medium

| | Stock | | Final | |
|---|---|---|---|---|
| Component | conc. | | conc. | |
| Advanced DMEM F-12 (Thermo | — | | To volume | |
| 12634028 ) | | | | |
| Penicillin Streptomycin (Thermo | 100% | | 1% | |
| 15140122) | | | | |
| L-Glutamine (Thermo 10378016) | 100 | X | 1 | X |
| B-27 supplement (Gibco 17504-044) | 50 | X | 1 | X |
| n-acetylcysteine (Sigma A9165) | 500 | mM | 1.25 | mM |
| R-spondin (conditioned medium Hans | 10 | mL | 10% | |
| Clevers Lab) | | | | |
| EGF (Thermo PMG8043) | 500 | μg/mL | 50 | ng/mL |
| Gastrin (Sigma G9020) | 100 | μM | 10 | nM |
| FSK stock | 10 | μL | 10 | μM |
| FGF10 (Peprotech 100-26) | 100 | μg/mL | 100 | ng/mL |
| HGF (Peprotech 100-39) | 20 | μg/mL | 25 | ng/mL |
| TGFb inhibitor (A83-01) (Sigma | 5 | mM | 50 | μM |
| SML0788) | | | | |
| Primocin (Thermo) | 50 | mg/mL | 100 | μg/mL |

| | | | |
|---|---|---|---|
| Nicotinamide (Sigma 72340) | 1M | 10 | mM |

TABLE 6
Human hepatic organoid medium

| | Stock | | Final | |
|---|---|---|---|---|
| Component | conc. | | conc. | |
| Advanced DMEM F-12 (Thermo | — | | To volume | |
| 12634028 ) | | | | |
| Penicillin Streptomycin (Thermo | 100% | | 1% | |
| 15140122) | | | | |
| L-Glutamine (Thermo 10378016) | 100 | X | 1 | X |
| B-27 supplement (Gibco 17504-044) | 50 | X | 1 | X |
| R-spondin (conditioned medium Hans | 10 | mL | 15% | |
| Clevers Lab) | | | | |
| EGF (Thermo PMG8043) | 500 | μg/mL | 50 | ng/mL |
| n-acetylcysteine (Sigma A9165) | 500 | mM | 1.25 | mM |
| Gastrin (Sigma G9020) | 100 | μM | 10 | nM |
| Nicotinamide (Sigma 72340) | 1M | | 10 | mM |
| FGF7 (Peprotech 100-19) | 50 | μg/mL | 100 | ng/mL |
| FGF10 (Peprotech 100-26) | 100 | μg/mL | 100 | ng/mL |
| HGF (Peprotech 100-39) | 20 | μg/mL | 50 | ng/mL |
| GSK-3 inhibitor (CHIR 99021) | 3 | mM | 3 | μM |
| (Tocris 4423) | | | | |
| TGFa (Peprotech 100-16A) | 20 | μg/mL | 50 | μg/mL |
| Primocin (Thermo) | 50 | mg/mL | 100 | μg/mL |

Culture of Human Fetal Small Intestinal Organoids and Pancreatic Ducts

Small intestines and pancreases were dissected from human fetal tissue fragments obtained immediately after termination of pregnancy from 10 to 20 PCW (post conception week), in compliance with the bioethics legislation in the UK. Fetal samples were sourced via the Joint MRC/Wellcome Trust Human Developmental Biology Resource under informed ethical consent with Research Tissue Bank ethical approval (08/H0712/34+5 and 08/H0906/21+5). For small intestines, similar isolation protocol for the mouse small intestine was adopted. For the pancreases, mesenchyme surrounding the pancreas was removed and epithelial tissue was digested in dispase II (Gibco) in Hank's balanced salt solution (HBSS; Thermo Fisher) at 37° C. for 3 min. Further dissociation was performed using collagenase P (Sigma Aldrich) with gentle pipetting. Cell clusters were rinsed once with 4 mL of advanced Dulbecco's modified Eagle's medium/nutrient mixture F12 with 1% Penicillin/Streptomycin (AdDMEM/F12; +1% P/S) and several times with DMEM/F12 +1% P/S, mixed with 30 μL of Matrigel (100% concentration), and seeded in 24-well plates. For media recipes look in table 3 and 7.

TABLE 7
Human fetal pancreatic organoid medium

| | Stock | | Final | |
|---|---|---|---|---|
| Component | conc. | | conc. | |
| Advanced DMEM F-12 (Thermo | — | | To volume | |
| 12634028 ) | | | | |
| Penicillin Streptomycin (Thermo | 100% | | 1% | |
| 15140122) | | | | |
| L-Glutamine (Thermo 10378016) | 100 | X | 1 | X |
| B-27 supplement (Gibco 17504-044) | 50 | X | 1 | X |
| N-2 supplement (Gibco 17502-048) | 50 | X | 1 | X |
| Nicotinamide (Sigma 72340) | 2M | | 10 | mM |
| N-Acetyl-L-cysteine (Sigma A9165) | 1 | mM | 1 | μM |
| R-spondin 1 (Peprotech 120-38) | 100 | μg/mL | 500 | ng/mL |
| Noggin (Peprotech 120-10C) | 100 | μg/mL | 100 | ng/mL |
| EGF (Peprotech AF-100-15) | 50 | μg/mL | 50 | ng/mL |
| FGF-10 (Peprotech AF-100-26) | 100 | μg/mL | 100 | ng/mL |
| Exendin4 (Sigma E7144) | 100 | μM | 100 | nM |
| Gastrin (Sigma G9020) | 100 | μM | 100 | nM |

Passage of Organoids in ECM Gel and Matrigel

Cell were passaged every 6-8 days. To passage the organoids, ECM gel and Matrigel droplets are thoroughly disrupted by pipetting in the well and transferred to tubes in ice. Cells are washed with 10 mL of cold basal DMEM F-12+++(F-12+P/S+HEPES+Glutamax) and spin at 200 g at 4° C. Supernatant is discarded. If any ECM or Matrigel is left, wash is repeated. The pellet is resuspended in 1 mL of cold basal medium and organoids are manually disrupted by narrow (flamed) glass pipette pre-wet in BSA 1% in PBS, to avoid adhesion to the glass. Cells are washed, pelleted and supernatant is discarded. Almost-dry pellets of disaggregated organoids are included either in cold liquid Matrigel or in cold ECM equilibrated gel, aliquot in 30-40 μL droplets in Petri dishes and incubated at 37° C. for 30 minutes to form a gel. For single cell colony formation and for monolayer cell seeding, organoids pellets are treated with TrypLE Express for 5-7 min (depending on organoid size and type) at 37° C. and accurate pipetting. Disaggregated cells are washed, pelleted, and resuspended in culture medium with ROCK inhibitor. Media recipes are reported in table 2-7.

In Vivo Implantation

Animal work was ethically approved and carried out under Home Office Project Licence PPL PDD3A088A. NODSCID-gamma (NSG) mice were anaesthetized with a 2-5% isoflurane:oxygen gas mix for induction and maintenance. Pancreas organoids were embedded in ECM gel and Matrigel drops within sterile silicon O-rings (3.35×1.20). Cultures were conducted for 2-4 days before grafting subcutaneously of NSG mice. For subcutaneous transplantation, buprenorphine 0.1 mg/Kg was administered at the induction for analgesia. Under aseptic conditions a midline incision (0.5 cm) was performed on the back of the mice and the ECM gel and Matrigel drops within the O-rings were inserted in lateral pockets. Mice were sacrificed at 2.5 weeks, 4 weeks, and 8 weeks post-transplant and content of the rings fixed in 4% PFA for 1 h for histological analysis.

Quantification of Stem Cell Colonies and Organoid Diameters

To quantify the colony formation in ECM gels and Matrigel, n≥10 fields of view at 5X per replicate were acquired at the Zeiss Axio Observer Al and counted. For organoid dimension quantification, n≥30 full grown organoids were randomly quantified in different 5X fields of view per replicate. For a better approximation, 3 diameters per organoid were measured and mean diameter was considered in the final calculation.

Cell Viability Assay

Cells were passaged to ECM gel and Matrigel and seeded in quadruplicates. Enteroids were expanded for 2 days and tested for combined gel cytocompatibility. Vitality assay was performed using LIVE/DEAD™ Viability/Cytotoxicity Kit, for mammalian cells (Thermo Fisher), following supplier instructions. Briefly, organoids were washed with basal DMEM F-12 and incubated in basal medium with hoechst, calcein-AM and ethidium homodimer-1 for 45 minutes. Cells in ECM gel and Matrigel droplets were washed twice and analyzed. Hepatic organoids vitality was analyzed through Cell Titer-Glo viability assay (Promega) following manufacturer's instructions.

Immunofluorescence and Protein Quantification

ECM gel and Matrigel droplets with embedded enteroids were fixed in 2% glutaraldehyde dissolved in PBS with Ca/Mg for 1 h at room temperature, and then washed. For sections, droplets were dehydrated with sucrose 30% overnight, included in OCT and cut at the cryostat microtome in 7 μm sections. Whole mount staining was performed by blocking and permeabilizing the cells with PBS-Triton 0.5% with BSA 1%. Primary antibodies were incubated in blocking buffer for 24h at 4° C. in rotation and extensively washed. Secondary antibodies were incubated overnight at 4° C. in rotation and washed. Antibody list and dilutions are reported in table 8.

TABLE 8
Antibody and molecule list

| Antibody/conjugated molecule | Dilution | |
|---|---|---|
| Ezrin (Thermo PA5-29358) | 1:100 | |
| rFABP1 (R&D AF1565) | 1:100 | |
| FITC-conjugated B4 isolectin (BSI-B4; Griffonia | 20 | μg/mL |
| (Bandeiraea) simplicifolia) (Sigma L2895) | | |
| alpha-Gal (M86) (Enzo ALX-801-090-1) | 1:5 | |
| GFP (Thermo A-21311) | 1:100 | |
| E-cadherin (BD 610182) | 1:100 | |
| Muc-1A (Termo HM-1630-P0) | 1:200 | |
| PDX-1 (Abacm AB47308) | 1:200 | |
| Sox-9 (Merk AB5535) | 1:500 | |
| Ki-67 (ABCAM Ab15580) | 1:200 | |
| Lysozyme (Genetex GTX72913) | 1:100 | |
| Lysozyme (Genetex GTX39779) | 1:100 | |
| Mucin-2 (Genetex GTX100664) | 1:100 | |
| Villin (Genetex GTX109940) | 1:100 | |
| Olfactomedin-4 (Cell signaling 14369S) | 1:50 | |
| Cytokeratin-20 (Proteintech 60183-1-lg) | 1:100 | |
| Zonula occludens-1 (Invitrogen 40-2200) | 1:200 | |
| Mucin-5AC (Thermo MA5-12178) | 1:100 | |
| Cytokeratin-19 (Abeam AB76539) | 1:100 | |
| Phalloidin 488 (Thermo A12379) | 1:200 | |
| Goat anti-Rabbit 594 (Thermo A11012) | 1:500 | |
| Goat anti-Rabbit 568 (Thermo A11011) | 1:500 | |
| Goat anti-Rabbit 488 (Thermo A11008) | 1:500 | |
| Goat anti-Mouse 488 (Thermo A11001) | 1:500 | |
| Goat anti-Mouse 568 (Thermo A10037) | 1:500 | |
| Donkey anti-Goat 647 (Thermo A-21447) | 1:500 | |
| Anti-Guinea pig (Jackson 706-165-148) | 1:500 | |
| Anti-Hamster (Abeam AB175716) | 1:500 | |
| Hoechst 33342 (Thermo H1399) | 10 | μg/mL |
| Calcein-AM (Thermo L3224) | 3 | μM |
| Ethidium homodimer-1 (Thermo L3224) | 3 | μM |

For human albumin protein quantification, human ductal organoids and human fetal hepatic organoids were culture for 3 days (with no medium change) and medium was collected from each well (n=4 per condition). Spent media were analyzed with Human Albumin ELISA (enzyme-linked immunosorbent assay) kit, following manufacturer's instructions.

Image Acquisition

Mouse and human organoids were imaged using a Zeiss Axio Observer Al. Stained sections were acquired on a Leica DMIL microscope and DFC420C camera, or using a Hamamatsu Photonics NanoZoomer. Immunofluorescence images of whole mount stainings and sections were acquired on a confocal microscope Zeiss LSM 710.

Bulk 3′ RNA-Sequencing

For human pediatric small intestinal organoids, RNA was isolated from cultured organoids in ECM gel and Matrigel with 20 min treatment of the droplets with Cell Recovery Solution (Corning) at 4° C. Cells were then washed in ice cold PBS to remove matrix leftovers that could interfere with RNA isolation. Organoids were centrifuged at 200 g at 4° C. and surnatant discarded. Dry pellet was lysed with RLT buffer (Qiagen). RNA was isolated with RNeasy Mini Kit (Qiagen) following manufacturer's instructions. Total RNA (100 ng) from each sample was prepared using QuantSeq 3′ mRNA-Seq Library prep kit (Lexogen GmbH) according to manufacturer's instructions. The amplified fragmented cDNA of 300 bp in size were sequenced in single-end mode using the Nova Seq 6000 (IIlumina) with a read length of 100 bp.

For human liver ductal organoids, and human fetal hepatic organoids, 5 ng of RNA/sample were used as input for the library preparation following the CEL-Seq2 technique as previously described⁴⁹.

Transcriptome Bioinformatic Analyses

For human pediatric small intestinal organoids, Illumina novaSeq base call (BCL) files were converted into fastq files through bcl2fastq (version v2.20.0.422) following software guide. Sequence reads were trimmed using bbduk software (bbmap suite 37.31), following software guide, to remove adapter sequences, poly-A tails and low-quality end bases (regions with average quality below 6). Alignment was performed with STAR 2.6.0a⁵⁰ on hg38 reference assembly obtained from cellRanger website (Ensembl 93), following online site guide. The expression levels of genes were determined with htseq-count 0.9.1 by using cellRanger pre-build genes annotations (Ensembl Assembly 93). All transcripts having <1 CPM in less than 4 samples and percentage of multimap alignment reads>20% simultaneously were filtered out. Differentially expressed genes (DEGs) were computed with edgeR⁵¹, using a mixed criterion based on p-value, after false discovery rate (FDR) correction by Benjamini-Hochberg method, lower than 0.05 and absolute log 2(fold change) higher than 1. A Principal Component Analysis was performed by Singular Value Decomposition (SVD) on log 2(CPM+1) data, after centering, using MATLAB R2019a (The MathWorks). Hierarchical clustering of ECM-related gene sets²⁷ was performed with Euclidean distance and complete linkage using median-centered data, and plotted as heat maps using MATLAB. DEGs over-representation analysis of Gene Ontology (GO) categories was performed using ClueGO (version 2.5.4)⁵².

For human liver ductal organoids, and human fetal hepatic organoids DNA library sequencing, mapping to the human reference genome and quantification of transcript abundance were performed as previously described⁴⁹. Sequencing libraries were analyzed using the DESeq2 package⁵³ in R version 3.4.0 and RStudio version 1.0.143.

Real Time PCR

cDNA was prepared using High-Capacity cDNA Reverse Transcription Kit (Applied Biosystems, #4368813). Quantitative PCR detection was performed using PowerUp™ SYBR® Green Master Mix (Applied Biosystems, A25742). Assays for each sample were run in triplicate and were normalized to housekeeping gene β-actin, where data was expressed as Mean±SEM.

Stiffness Measurement

Stiffness measures were taken at the Piuma Nanoindenter (Optics 11) on Petri-dishes with 30 μL ECM gels and Matrigel droplets immersed in PBS. The probe parameters used for the measures were: tip radius 57 μm, and probe stiffness 0.44 N/m.

Co-Polymerized Hydrogels

Polyacrylamide pre-polymer is prepared by mixing acrylamide/bis-acrylamide, 40% solution 29:1 (Sigma Aldrich) with PBS −/− and photo-initiator irgacure 2959 (Ciba) solved at 35 mg/mL in methanol (Sigma Aldrich). For 1 mL of a 20% final acrylamide concentration, 100 μL of irgacure, 500 μL acr/bis-acr solution and 400 μL of PBS are mixed and kept in the dark until use. Neutralized 10 mg/mL ECM pre-gel is allowed to gelate in incubator for 30 min. The gel is then disaggregated by repetitive pipetting and thoroughly mixed with polyacrylamide pre-polymer with proportions 25-75, 50-50, 75-25. Liquid pre-gel is then polymerized between two cover glasses and a silicon ring by photoactivation at the DYM40183 BlueWave 75 UV curing spot lamp. Co-polymerized hydrogel is then extensively washed in PBS with Pen-Strep to remove any cytotoxic acrylamide monomer from the gel bulk. Prior to use for cell seeding, co-polymerized hydrogels are cut and positioned in culture wells, and pre-equilibrated with basal medium overnight.

Statistical Analysis

Statistical analyses were performed using the following software: Matlab (v. R2017a) for PCA, pie plot, bar plot, hierarchical clustering with proteomic and RNA-seq data, Microsoft Excel Professional Plus (v. 2016 MSO) to normalize and filter the proteomic data, GraphPad Prism Mac (v. 6.0h) was used with all other graphs and charts.

### REFERENCES

1. Fatehullah, A., Tan, S. H. & Barker, N. Organoids as an in vitro model of human development and disease. Nat. Cell Biol. 18, 246-254 (2016).
2. Barker, N. et al. Identification of stem cells in small intestine and colon by marker gene Lgr5. Nature 449, 1003-1007 (2007).
3. Barker, N. et al. Lgr5+ve Stem Cells Drive Self-Renewal in the Stomach and Build Long-Lived Gastric Units In Vitro. Cell Stem Cell 6, 25-36 (2010).
4. Fukuda, M. et al. Small intestinal stem cell identity is maintained with functional Paneth cells in heterotopically grafted epithelium onto the colon. Genes Dev. 28, 1752-7 (2014).
5. Schwank, G. et al. Functional Repair of CFTR by CRISPR/Cas9 in Intestinal Stem Cell Organoids of Cystic Fibrosis Patients. Cell Stem Cell 13, 653-658 (2013).
6. Broutier, L. et al. Culture and establishment of self-renewing human and mouse adult liver and pancreas 3D organoids and their genetic manipulation. Nat. Protoc. 11, 1724-1743 (2016).
7. Fordham, R. P. et al. Transplantation of expanded fetal intestinal progenitors contributes to colon regeneration after injury. Cell Stem Cell 13, 734-744 (2013).
8. Yui, S., Azzolin, L., Schweiger, P. J., Piccolo, S. & Jensen Correspondence, K. B. YAP/TAZ-Dependent Reprogramming of Colonic Epithelium Links ECM Remodeling to Tissue Regeneration. Stem Cell 22, 35-49.e7 (2018).
9. Gjorevski, N. et al. Designer matrices for intestinal stem cell and organoid culture. Nature 539, 560-564 (2016).
10. Cruz-Acuña, R. et al. Synthetic hydrogels for human intestinal organoid generation and colonic wound repair. Nat. Cell Biol. 19, 1326-1335 (2017).
11. Calle, E. A. et al. Targeted proteomics effectively quantifies differences between native lung and detergent-decellularized lung extracellular matrices. Acta Biomater. 46, 91-100 (2016).
12. Gaetani, R. et al. Evaluation of Different Decellularization Protocols on the Generation of Pancreas-Derived Hydrogels. TISSUE Eng. Part C 24, (2018).
13. Dohmen, P. M. Clinical results of implanted tissue engineered heart valves. HSR Proc. Intensive Care Cardiovasc. Anesth. 4, 225-31 (2012).
14. VeDepo, M. C., Detamore, M. S., Hopkins, R. A. & Converse, G. L. Recellularization of decellularized heart valves: Progress toward the tissue-engineered heart valve. J. Tissue Eng. 8, 2041731417726327 (2017).
15. Yu, Y., Alkhawaji, A., Ding, Y. & Mei, J. Decellularized scaffolds in regenerative medicine. Oncotarget 7, 58671-58683 (2016).
16. Saheli, M. et al. Three-dimensional liver-derived extracellular matrix hydrogel promotes liver organoids function. J. Cell. Biochem. 119, 4320-4333 (2018).
17. Badylak, S. Endothelial cell adherence to small intestinal submucosa: an acellular bioscaffold. Biomaterials 20, 2257-2263 (1999).
18. Stephen F. Badylak, Sherry Voytik, G. B. Submucosa gel as a growth substrate for cells. Pat. 5,866,414 (1995).
19. Voytik-Harbin, S. L. E. E., Brightman, A. O., Waisner, B. Z., Robinson, J. P. & Lamar, C. H. Small Intestinal Submucosa: A Tissue-Derived Extracellular Matrix That Promotes Tissue-Specific Growth and Differentiation of Cells in Vitro. TISSUE ENGINEERING 4, (Mary Ann Liebert, Inc, 1998).
20. Totonelli, G. et al. A rat decellularized small bowel scaffold that preserves villus-crypt architecture for intestinal regeneration. Biomaterials 33, 3401-3410 (2012).
21. Manka, S. W. et al. Structural insights into triple-helical collagen cleavage by matrix metalloproteinase 1. Proc. Natl. Acad. Sci. U.S.A 109, 12461-6 (2012).
22. Quesenberry, P. J., Aliotta, J., Chiara Deregibus, M. & Camussi, G. Generation of disease-specific induced pluripotent stem cells from patients with different karyotypes of Down syndrome. (2012). doi:10.1186/s13287-015-0150-x
23. Huch, M. et al. Long-term culture of genome-stable bipotent stem cells from adult human liver. Cell 160, 299-312 (2015).
24. Hu, H. et al. Long-Term Expansion of Functional Mouse and Human Hepatocytes as 3D Organoids. Cell 175, 1591-1606.e19 (2018).
25. Daly, K. A. et al. Effect of the aGal Epitope on the Response to Small Intestinal Submucosa Extracellular Matrix in a Nonhuman Primate Model.
26. Barker, N. Adult intestinal stem cells: Critical drivers of epithelial homeostasis and regeneration. Nat. Rev. Mol. Cell Biol. 15, 19-33 (2014).
27. Hynes, R. O. & Naba, A. Overview of the matrisome-—an inventory of extracellular matrix constituents and functions. Cold Spring Harb. Perspect. Biol. 4, a004903 (2012).
28. Hughes, C. S., Postovit, L. M. & Lajoie, G. A. Matrigel: A complex protein mixture required for optimal growth of cell culture. Proteomics 10, 1886-1890 (2010).
29. Aizarani, N. et al. A human liver cell atlas reveals heterogeneity and epithelial progenitors. Nature doi:10.1038/s41586-019-1373-2
30. Tian, H. et al. A reserve stem cell population in small intestine renders Lgr5-positive cells dispensable. (2012). doi:10.1038/nature10408
31. Tibbitt, M. W. & Anseth, K. S. Hydrogels as extracellular matrix mimics for 3D cell culture. Biotechnol. Bioeng. 103, 655-63 (2009).
32. Sato, T. et al. Single Lgr5 stem cells build crypt-villus structures in vitro without a mesenchymal niche. Nature 459, 262-265 (2009).
33. Ashe, H. L. & Briscoe, J. The interpretation of morphogen gradients. Development 133, 385-94 (2006).
34. Nelson, C. M. & Bissell, M. J. Of Extracellular Matrix, Scaffolds, and Signaling: Tissue Architecture Regulates Development, Homeostasis, and Cancer. Annu. Rev. Cell Dev. Biol. is online Annu. Rev. Cell Dev. Biol 22, 287-309 (2006).
35. Simo, P. et al. Changes in the expression of laminin during intestinal development. Development 112, 477-87 (1991).
36. Wedgwood, J., Freemont, A. J. & Tirelli, N. Rheological and Turbidity Study of Fibrin Hydrogels. doi:10.1002/masy.201300111
37. Stokes, D. J. Principles and Practice of Variable Pressure/Environmental Scanning Electron Microscopy (VP-ESEM). (2008).
38. Lokman, N. A., Elder, A. S. F., Ricciardelli, C. & Oehler, M. K. Chick Chorioallantoic Membrane (CAM) Assay as an In Vivo Model to Study the Effect of Newly Identified Molecules on Ovarian Cancer Invasion and Metastasis. OPEN ACCESS Int. J. Mol. Sci 13, 13 (2012).
39. Wiśniewski, J. R., Zougman, A., Nagaraj, N. & Mann, M. Universal sample preparation method for proteome analysis. Nat. Methods 6, 359-362 (2009).
40. Cox, J. & Mann, M. MaxQuant enables high peptide identification rates, individualized p.p.b.-range mass accuracies and proteome-wide protein quantification. Nat. Biotechnol. 26, 1367-1372 (2008).
41. Schwanhausser, B. et al. Global quantification of mammalian gene expression control. (2013). doi:10.1038/nature10098
42. Huang, D. W., Sherman, B. T. & Lempicki, R. A. Systematic and integrative analysis of large gene lists using DAVID bioinformatics resources. (2008). doi:10.1038/nprot.2008.211
43. Wilhelm, M. et al. Mass-spectrometry-based draft of the human proteome. Nature 509, 582-587 (2014).
44. Schmidt, T. et al. ProteomicsDB. Nucleic Acids Res. 46, 1271-1281 (2017).
45. Fiehn, O. Metabolomics by Gas Chromatography-Mass Spectrometry: Combined Targeted and Untargeted Profiling. in Current Protocols in Molecular Biology 114, 30.4.1-30.4.32 (John Wiley & Sons, Inc., 2016).
46. Tsugawa, H. et al. MS-DIAL: data-independent MS/MS deconvolution for comprehensive metabolome analysis. Nat. Methods 12, 523-526 (2015).
47. Jung, P. et al. Isolation and in vitro expansion of human colonic stem cells. Nat. Med. 17, 1225-1227 (2011).
48. Sato, T. et al. Long-term expansion of epithelial organoids from human colon, adenoma, adenocarcinoma, and Barrett's epithelium. Gastroenterology 141, 1762-72 (2011).
49. Kretzschmar, K. et al. Profiling proliferative cells and their progeny in damaged murine hearts. doi:10.1073/pnas.1805829115
50. Dobin, A. et al. Sequence analysis STAR: ultrafast universal RNA-seq aligner. 29, 15-21 (2013).
51. Robinson, M. D., Mccarthy, D. J. & Smyth, G. K. edgeR: a Bioconductor package for differential expression analysis of digital gene expression data. Bioinforma. Appl. NOTE 26, 139-140 (2010).
52. Bindea, G. et al. ClueGO: a Cytoscape plug-in to decipher functionally grouped gene ontology and pathway annotation networks. Bioinforma. Appl. NOTE 25, 1091-1093 (2009).
53. Love, M. I., Huber, W. & Anders, S. Moderated estimation of fold change and dispersion for RNA-seq data with DESeq2. Genome Biol. 15, 550 (2014).

1. A method of preparing an extracellular matrix powder pre-gel solution (“ECM pre-gel”), the method comprising:
(a) providing decellularised tissue;
(b) processing the decellularised tissue to derive ECM powder from said tissue; and
(c) digesting the powder at a concentration of 1 mg/mL to 8 mg/mL in a proteolytic solution, thereby forming said ECM pre-gel.

2. The method of claim 1, wherein the decellularised tissue is intestinal tissue, optionally small intestinal tissue.

3. The method of claim 2, wherein the small intestinal tissue is the mucosal/submucosal layers of the small intestine.

4. The method of any of the above claims, wherein the powder is digested at a concentration of 2 mg/mL to 8 mg/mL, 2 mg/mL to 6 mg/mL, or at a concentration of 2 mg/mL to 4 mg/mL.

5. The method of any of the above claims, wherein, prior to step (c), the ECM powder is: sterilized by gamma radiation; and/or the decellularised tissue is washed in Milli-Q water and then washed with DNase.

6. A method of preparing an extracellular matrix powder gel solution (“ECM gel”) prepared by the following steps:
(i) performing the method of any one of claims 1 to 5,
(ii) neutralising the ECM pre-gel to form a gel solution with a pH of between 6.8 to 7.7, thereby forming said ECM gel.

7. The method of claim 6 wherein, prior to step (ii), the ECM pre-gel is centrifuged, and precipitated undigested pellets of decellularized tissue, that result from the centrifugation step, are discarded.

8. The method of claim 6 or claim 7, wherein, in step (ii), the ECM pre-gel is neutralised in Dulbecco's Modified Eagle Medium (DMEM).

9. An ECM pre-gel prepared by the method of any one of claims 1 to 5.

10. An ECM gel prepared by the method of any one of claims 6 to 8.

11. An organoid culture, comprising: organoids, pieces of organoids, or organoid cell pellets; and an ECM gel of claim 10.

12. A method of preparing an organoid culture, comprising mixing: organoids, pieces of organoids, or organoid cell pellets; and an ECM gel of claim 10; thereby forming said culture.

13. The method of claim 12, further comprising aliquoting droplets of the mixture onto a suitable medium, optionally where the suitable medium is a petri dish, and/or optionally wherein the droplets have a volume of 30-40 μL.

14. The culture or method of any one of claims 11-13, wherein the organoids or organoid cell pellets are endoderm-derived organoids or organoid cell pellets.

15. The culture or method of any one of claims 11-14, wherein the endoderm-derived organoids or organoid cell pellets are selected from:
organoids or organoid cell pellets of gastric origin; stomach enteroids or enteroid cell pellets;
pediatric stomach enteroids or enteroid cell pellets; ductal (cholangiocyte) organoids or organoid cell pellets; fetal hepatic (hepatocyte) organoids or organoid cell pellets; intestinal stem cells or stem cell pellets; Lgr5+ intestinal stem cells or stem cell pellets; organoid or organoid cell pellets of fetal origin; small intestinal enteroids or enteroid cell pellets; adult cholangiocyte ducts; fetal hepatocyte organoids or organoid cell pellets; human ductal organoids or organoid cell pellets; ductal liver organoids or organoid cell pellets; intestinal organoids or organoid cell pellets; or fetal pancreatic organoids or cell pellets.

16. The culture or method of any one of claims 11-15, wherein the organoids or organoid cell pellets are human or mouse organoids or organoid cell pellets.

17. The culture or method of any one of claims 11-16, wherein the organoid culture can be maintained for more than two passages.

18. The culture or method of any one of claims 11-17, wherein the organoid culture can maintain expression of essential markers after seven days from culture formation.

19. The culture or method of any one of claims 11-18, wherein the organoid culture can be maintained for more than one month from culture formation.

20. The culture or method of any one of claims 11-19, wherein the culture does not comprise Matrigel and/or basement membrane extract.

21. The culture or method of any one of claims 11-20, wherein gene expression in the organoids or organoid cell pellets of the culture is comparable to gene expression in an organoid or organoid cell pellet culture comprising Matrigel.

22. The culture or method of claim 21, wherein the gene is selected from the group consisting of: LGR5, OLFM4, SMOC2, LYZ, BMI1, LRIG1, FABP1, MUC1, MUC3A, MUC5B, EZR, VIL1, MUC12, MUC13, MUC17, MUC20, CHGA, TINAGL1, LTBP4, CRELD1, ECM1, LGALS1, LGALS3, LMAN1, P4HA1, KRT7, KRT8, KRT18, KRT19, EPCAM, SOX9, TACSTD2 (TROP2), ALB, ASGR1, ASGR2, SERPINA1, FABP1, APOA2, ALPI and MUC2.

23. A method of in vivo delivery of an organoid culture to a subject, comprising administering to the subject the culture of claim 11 or claims 14-22, or the culture obtained by the method of any one of claims 12-22.

24. A method of treatment of disease in a subject, comprising administering to the subject the culture of claim 11 or claims 14-22, or the culture obtained by the method of any one of claims 14-22.

25. The method of claim 23 or claim 24, wherein the culture is conducted for 2-4 days prior to administration.

26. The method of any of claims 23-25, wherein the subject is a human subject, a mouse subject, or a mouse model of disease.

27. The method of any of claims 25-26, wherein the organoid is a human fetal pancreatic organoid or small intestinal organoid.

28. The method of any of claims 23-27, wherein organoid organisation is preserved after 2 weeks, after 3 weeks, after 4 weeks, after 5 weeks, after 6 weeks, after 7 weeks, or after 8 weeks from administration.

29. The method of any of claims 23-27, wherein the culture comprises matured organoids after 2 weeks, after 3 weeks, after 4 weeks, after 5 weeks, after 6 weeks, after 7 weeks, or after 8 weeks from administration.

30. The method of any of claims 23-29, wherein gene expression of the organoid is comparable to gene expression in to an organoid culture comprising Matrigel.

31. The culture of claim 11 or claims 14-22, or the culture obtained by the method of any one of claims 12-22, for use in the method of any of claims 23-30.

32. Use of the culture of claim 11 or claims 14-22, or the culture obtained by the method of any one of claims 12-22, in the manufacture of a medicament for the method of any of claims 23-30.

33. An organoid culture comprising: organoids, pieces of organoids or organoid cell pellets; and an extracellular matrix powder gel solution (“ECM gel”) comprising ECM powder at a concentration of between 1 mg/mL and 8 mg/mL.

34. The culture of claim 33, wherein the organoids or organoid cell pellets are endoderm-derived organoids or organoid cell pellets.

35. The culture of claim 33 or claim 34, wherein the endoderm-derived organoids or organoid cell pellets are selected from-organoids or organoid cell pellets of gastric origin; stomach enteroids or enteroid cell pellets; pediatric stomach enteroids or enteroid cell pellets; ductal (cholangiocyte) organoids or organoid cell pellets; fetal hepatic (hepatocyte) organoids or organoid cell pellets; intestinal stem cells or stem cell pellets; Lgr5+ intestinal stem cells or stem cell pellets; organoid or organoid cell pellets of fetal origin; small intestinal enteroids or enteroid cell pellets; adult cholangiocyte ducts; fetal hepatocyte organoids or organoid cell pellets; human ductal organoids or organoid cell pellets; ductal liver organoids or organoid cell pellets; intestinal organoids or organoid cell pellets; or fetal pancreatic organoids or cell pellets.

36. The culture or method of any one of claims 33-35, wherein the organoids or organoid cell pellets are human or mouse organoids or organoid cell pellets.

37. The culture or method of any one of claims 33-36, wherein the organoid culture can be maintained for more than two passages.

38. The culture or method of any one of claims 33-37, wherein the organoid culture can maintain expression of essential markers after seven days from culture formation.

39. The culture or method of any one of claims 33-38, wherein the organoid culture can be maintained for more than one month from culture formation.

40. The culture or method of any one of claims 33-39, wherein the culture does not comprise Matrigel and/or basement membrane extract.

41. An extracellular matrix powder gel solution (“ECM gel”) prepared by the following steps:
(a) providing decellularised tissue;
(b) processing the decellularised tissue to derive ECM powder from said tissue;
(c) digesting the powder at a concentration of 1-20 mg/mL, or optionally 2-20 mg/mL, or optionally 5-15 mg/mL, or optionally 10 mg/mL, in a proteolytic solution, thereby forming an ECM pre-gel;
(d) neutralising the ECM pre-gel to form a gel solution with a pH of 6.8 to 7.7, thereby forming said ECM pre-gel; and
(e) allowing the ECM pre-gel to gelate; and
(f) mixing the ECM pre-gel with synthetic pre-polymer.

42. The ECM gel of claim 41, wherein the synthetic pre-polymer comprises poly-acrylamide.
